SEQUENCE LISTING (4 sequences)

SEQ ID NO: 1 - DNA, 5593 bp, Homo sapiens
  source 1..5593: mol_type = genomic DNA
gtctgaggcggcgacgagtggtaggagtcttctttcccctccttcccctttcccttcccttcccgggagaggctgggacccggcaccagggcagtactgtggccgctgcggcctcagctccgactgggtcaggttgcggagactccaggccgcttccagggcgagtactcctgattgtgacatcacattcatcccctgggcgatggagcttgtcactgggaaggaatactcagtcggagaatagccaacaagatgggttactgggagaatctcttcagtggcactgagtggaggcatcagggggttggagccttgtgaacagggaacctgccccccaacacttggaaggacctgggtttcagtgatgagacatggggtatgatgtaacccgtttccagggggatgttgacgaagatcttatctgccctatttgcagtggagtcttggaggagccagtacaggcacctcattgtgaacatgctttctgcaacgcctgcatcacccagtggttctctcagcaacagacatgtccagtggaccgtagtgttgtgacggtcgcccatctgcgcccagtacctcggatcatgcggaacatgttgtcaaagctgcagattgcctgtgacaacgctgtgttcggctgtagtgccgttgtccggcttgacaacctcatgtctcacctcagcgactgtgagcacaacccgaagcggcctgtgacctgtgaacagggctgtggcctggagatgcccaaagatgagctgcccaaccataactgcattaagcacctgcgctcagtggtacagcagcagcagacacgcatcgcagagctggagaagacgtcagctgaacacaaacaccagctggcggagcagaagcgagacatccagctgctaaaggcatacatgcgtgcaatccgcagtgtcaaccccaaccttcagaacctggaggagacaattgaatacaacgagatcctagagtgggtgaactcccttcagccagcaagagtgacccgctggggagggatgatctcgactcctgatgctgtgctccaggctgtaatcaagcgctccctggtggagagtggctgtcctgcttctattgtcaacgagctgattgaaaatgcccacgagcgtagctggccccagggtctggccacactagagactagacagatgaaccgacgctactatgagaactacgtggccaagcgcatccctggcaagcaggctgttgtcgtgatggcctgtgagaaccagcacatgggggatgacatggtgcaagagccaggccttgtcatgatatttgcgcatggcgtggaagagatataagagaactcgactggctatcaggaagagatggaaatcagaaaatcccatcactccagcagctgggacctgagtcctacccaccattcttaatactgtggcttatacctgagccacacatctccctgcccttctggcactgaagggccttggggtagtttgctcagcctttcaggtgggaaacccagatttcctccctttgccatattcccctaaaatgtctataaattatcagtctgggtgggaaagcccccacctccatccattttcctgcttagggtccctggttccagttattttcagaaagcacaaagagattcaatttccctggaggatcaggacagaggaaggaatctctaatcgtccctctcctccaaaaccagggaatcagagcagtcaggcctgttgactctaagcagcagacatcctgaagaaatggtaagggtggagccaaatctctagaaataagtagtgaggccgttaattggccatcactgatggcccttagggaaagactggacctctgtgccaagcagtatccctgttcagcccaccttaaaggtgtaggcacccactgggtctaccagtatgcaggttgggatactgaaaatttccagatgagctcttctttcctacaagttttcataattagggaatgccagggtttagggtaggggttaatctgttgggggttgatgtgtttagcaagaagctactcctagcttttgctaaaatatggttggcactgcctcttgtggcacaggccataattgttccatagacccctctctagccctgtgactgtagttagttactttgataattttctttggccattgtttgtttatatttcacaaactccacctactgcccccccccctcttttttttaagaatggcctgatcatggctatctcagccacattgttggcaatttaatttatttacttcctttttttttttttaagaaaggaaaaaagaaaaaaaaatcaaacttgaaacttttcttttgatgttcctattgtgggggttctggatagggtgggacagggatgggggtgtgttttatattttttccttttcagcacaacctttggctttaatataggaagagccaagggagtcctcggctgaacttacgatatctgccccaaacctctgtaaccccaactgaaatgaggagcttcctctcttcctgtgaaggatatgacagtccagcatcgatgcctgtgccctctggaaaaatttcctcctagcccttccagggccttatcataaaactctggatttagagtattcattttgaaggcaactcccccttccccaagtttccttggagctgtatagctgggttctaagcttcaccatgcaaatcagaaattttatctctaagtacaggctgtgccgtgtctcacccacacccccctggggacttcagttccatttcaggttacctggggtataccttgatccctagagtgactggcagagtaagagaaggggagagataataggtgtgattattttaatatggaggtgggagtgtggttggagatagaaaggctcctccccaccatgtaatggcttcctctcagaattttattccaggctagcttgctgcaggtctgggtagttggatcatggctccactgggattggggtggaaagcttgaggggagtagggttccagctctgggacattgtgctcaggaatttgaaaacgctgctatacttactctggttactacatttcttccactcccctttcccctacctgccttaaccaaggctcatactgtcctgtccttaccctcagatggagccaggaagctcagtgaaaggcttccctaccctttgcactagtgtctctgcaggttgctggttgtgttgtatgtgctgttccatggtgttgactgcactaataataaaccttttactcaactctctaaattcttcagcattactccctttcttgagaaggtttcccctctgcttttgcctttctctcaccttaattccctttcttccttactttgttacctacccttatcttagtgctaacttctctttcaggaggatgtctgggagtagtgtgcacttcacagctgctttcccatgtaccctcctgcattcttccctcctatctcctgttctgtagcagccaaagctctctagtgatctgaactgtgtgcttcccagggtctgcctttatcctaaattccatgtcttccctgagtggtcctgagtttttgggataatttctacagaagatatgtatatatctttttcctttgtcccacaagcaactttgctttagaatctagaattcctttgcaggcagagaagtctctacctcccagtgtttcctagctaagaacgtaaatgtgaggagggaaatgtacttgcagaggtttcataattatttacttataaaaatagtcttcatagccgggcgcggtggctcacgcctgtaatcccagcactttgggaggccgaggtgggtggatcacaaggtcaggagttcgagaccatcctggctaacacagtgaaaccccgtctctactaaaaatacaaaaaattagccgggcgtggtggcaggcacctgtagtcccagctacttaggaggctgaggcaggagaatggcgtgaacccgggaggcagagcttgcagtgagcagagattgggccactgcattccagcctgggcgacagagcaaggctccgtctaaaaaaaaaaaaaaaaaaaaaagtcttcataggccgggcacggtggctcacgtctgtaatcccagcactttgggaggccaaggtgggtggatcacaacgtcaggagatcgagaccatcctggctaacatggtgaaaccctgtctctactaaaaatataaataaattagccggacaggcgcctgtcctcccagctactcaggaggctgaggcaggagaatggtgtgaacctgggaggcggagcttgcagtgagctgagatcacgccactgcactccagcctgggcaacagagcaagactccgtctcaaaaaaaaaaaaaaaaaaaccagtcttcataagtatttgctgctacctttccctgtcataagaaaaaggatagccagacatggtgggacgccactatgatcccagctccttggaaggctaaggcacaagaatcgcttgaacctgggaggtggaggttgcagtgagctgagatcatgccactgcactccagcctggtgacagagcaagagcctgtctcaaaaaaaaaaaagaaaagaaaagaaaaagggatatcttttcctcctcccagaagtttgttttaaatttgagcatttatcatgcacctgatgtaaacctaatagtactcttgatactctagtggcttgaaaaaaaaaaaaaaggcatttctgtgctgagtctgcgcttctatgcacacaaggtatgtttataaaatactgataagcatgtcacagtatagagcataagaggcaatgtatgtatcctagtgacattagcagtgcttttccccccttaaactcctttaaaattacttttagaacttgctgctcattcttgtgaatgttatgaatggtgtcatattgtccttttacagaagatacgatttttagaaacaaatattcattgaatgtctgccctgtgagatactcactagagtgaacatgaggaggcttatgtagcaaaatggcacctacctgcaaagaacttagtccctaatggagatgaatatataataagggatcataaatgtgctaagtggatttactagtaatatgtgagccaaggacgataaagctcctgattctgatgggtatcaggaaaggcttttcaggaagtgttacttgttataggtcagaggtcagcaaactacaggttacaaccccactgcctgcttttgtaaaaaactttattggaatacagttatgcccacttgtttatatattgtctgtggctgctttcacattacaacagcagggttaagtagctggaacaaagcctaaaatacttggtcttttacagaaaaaaatttgccaacctgttctaaacttttaaggtcaggagaactgggggaaggagggagtagaaagaacactgtcacccagactggagtgcagtggcaccatcatatagctcactgtagcctcaaactcctgggctctagtggtcttcccacttcagcttctggagtagctggggctactgcacctggaattgtcttaatctgttttaatactagtaaaattttttttgtttcatctctttca

SEQ ID NO: 2 - protein, 317 aa, Homo sapiens
MGYDVTRFQGDVDEDLICPICSGVLEEPVQAPHCEHAFCNACITQWFSQQQTCPVDRSVVTVAHLRPVPRIMRNMLSKLQIACDNAVFGCSAVVRLDNLMSHLSDCEHNPKRPVTCEQGCGLEMPKDELPNHNCIKHLRSVVQQQQTRIAELEKTSAEHKHQLAEQKRDIQLLKAYMRAIRSVNPNLQNLEETIEYNEILEWVNSLQPARVTRWGGMISTPDAVLQAVIKRSLVESGCPASIVNELIENAHERSWPQGLATLETRQMNRRYYENYVAKRIPGKQAVVVMACENQHMGDDMVQEPGLVMIFAHGVEEI

SEQ ID NO: 3 - DNA, 4394 bp, Mus musculus
  source 1..4394: mol_type = genomic DNA
gagcttccgccccgttgcgtagcagcagtgggcgtggctaagggctaacccgagacagcgaggagtggtcggaaccttcttccccatccttaccttccccttcccctacggggaaaggctgagaccgggcacacagtgtgctacgcggccgccgtgtcctcagctgcagccaggccccgcgtccggagaaaccaggctgcgttccgggtgagcgttcctgattgtgacatcacacccacctctgggccatgagacttgtcactaggaagaaagactcagttggagaatagccaacaagatgggatactcagagcatctcctgagcggcactgagcggaggcaccagggggttggaaccttgtgaacagggaacctgcccccaccacttaaaaggacctgggtttcagtgatgagacatggggtatgatgtgacccgtttccagggggacgttgacgaggaccttatctgccctatttgcagtggagtcttggaggagccagtgcaggcgcctcattgtgagcatgccttctgcaacgcctgcatcacccaatggttctctcagcagcagacgtgtccggtagaccgtagcgttgtcacggttgcccacctgcgcccagtacctcggatcatgcggaacatgttgtcaaagctacagattgcctgtgacaacgctgtgtttggctgcagtgctgttgtcaggcttgacaacctcatgtcccacctcagtgactgtgagcacaacccgaagcggcctgtgacctgtgagcagggctgtggcctggagatgcccaaagatgaactgccaaaccacaattgcattaagcacctgcgctccgtggtccagcagcagcagtcgcgcattgcagagctggagaagacctcggctgaacacaagcaccagctggcagagcagaagcgagacattcagctgctgaaggcgtatatgcgagccatccgcagtgtcaaccccaaccttcagaacctggaggagacaatcgaatacaatgagatcctcgagtgggtgaactccctgcagccggcaagggtgacccgctgggggggcatgatctccactcctgatgctgtgctccaggctgtcatcaagcgctccctcgtggaaagtggctgcccggcctccatcgtcaacgagctgattgaaaatgcccatgaacgcagctggccccagggtctggccacactagagacaagacagatgaaccggcgctactatgagaactacgtggccaagcgcatccctggcaagcaggctgtagtggtgatggcctgtgagaaccagcacatgggggacgacatggtgcaggagccagggctcgtcatgatatttgcgcatggtgtggaggagatataagagcgtaactggctgtcaggaagaggtagagttggaaaacccattgctccagcagctgagccctgacttctcccacagcccttatgccgcggcttgtacctgagccacgtactccctgcttctggcatgaaggactttggggcacttagctcagcctttcagcggagaacccagattcctgccttttgctatattctttcccccacaatgtctgtaaattttcagtcagggtggagaaatccccacttctgtattttcctgactaagatcccctggttcccaaatattttcagaaagcacaaagaaatttgttttccctagaggatcagggtagagtgaggcatttctaattgtctatctcttccaaaaccagggagtcccagcaggcaggagtcccagcaggcaggcttgctgaagtcacgcagcattgagagctgctctgaggagccagcacctggcagaagggctgagggtgtggcccaaaccctctaaccagtgaggccaaagcctggtcctcacgcgcatccctgtccaggccgctgtgaaggtgcaggccttgatgggtgcgcagggtggcccctgtgagggtgcacagggtgggttgttgaaatgttgctgaactctttcctacacgtttcataattaggaagggaaggaaggggtgggcgtgagagcactggtgttcgtacactcagcaggaagctgctgtctggctctgctgatccagaacggcactgcctcaagagggtgtctgtgttgcctcggtggcccctcttcagccctgtgattgcagttagcttgatttcctttggtgttgtttgtttatatttccaagtcacaaaccccatctgttacccagtcttgttttcataggatggactggctgtgtctctctgatctagcccagccacattgttggcaatttaatttatttactttttttttttttaagaaaggagaaaagaaaaaacatcaagaaaacttaaaacctttcttatgccctttctttggctgttcctattgtgggatttgggtagggtggggctggtaaggtctgtttcatgtgtttccttttcagcatgagctctggtctgatacctgctctgaccctcccctccatcaccctgcaggagctttggctgtggcagtccagcgttgatgcctgtgacccatgaagaaatttgttccctgcccctttaggtctccttgtgaaaccctagaattaactggctggcacatgtgttttgaaggtgactcttccatgtttcctgaggaaacttcctgtttatctgggttctaaccttcaccagccaagtgggatataaatcagatgtctccaaagccaggctgactacactggctcagtcaagtccacccaaggacttggggtccattcaaggctctctgggtggctgctgggtcccccatggtggctagtagagtgagcaaggagaaagagtggtgtgattattttaaaagcgtaggtgggagtgtgattggagaggtcagagggccccccttcaccatactctagcttctctctgggagttccacttcaggctagcctgctgttggtctcactggttgagtcgtgtgcctcctgtggggttgggatagaaggatatagcttgaggaccgtaagaaaaagttgggtgttccagctccaggacattgtgctcaggagtttgaaaaaggctgctatactcagtcgggttactgggtttcttgttcctctttgtgcctgcctgccttgcccaagctcactctgtctattccttactctgagatgaatgatggaggaggctgaagtttgcttgccctttgcaccagtatctgcagctcttccattgtgctgtgtgctgtacatggtgttcactgcactaataaatctctcactcaactctaaattcttagtcaccccccttatgaaggattcttcctagcttggtaatgcaccttccactgacctctgccttgagagaccgtctgtctggaaactgcatacacttcacacgcttcacgttctgtgctctactgctctcgtaagcctaactgcattctcttcagagtgtccctttagccggaattctgttttcctaaatgtcctgcattcggaggatgctaccgaagactttagcacctctttccagcgagtggggagcttctgccctctaggggctcctgatattaatgtagggaggaaacctgaaacagctctcctgctgttcagaccttgacaaggactttcctggctagggagatggctagggagaggtaagagctctaactgcacgtgaggatgtgagttcaaattcccagcacctgcataaaaagctgagcatggtcaagagataactccctcgttgtgtggggcagaggtagagggcttactggagcttgctggtccctatttgagctccaagttcagtgactgacagtgtctcaaagaatgaagccaggtatggtggaacatgcctttaatcccagtactttggagccagcagacagatctcagtgaggggcctgcaagtttaaaggaatatggcatagtaataaaacaggacacccaacatcctcttccaaacaccttgaaagcatgagtatgcacaatgcacatacatgtccaccatgctcacacatttttgctgaacactttttgttactatcttattcctcctatgagaaaccatgtgtgtgtgtgtgtgtgtgtgtgcatgtgtgacttaagatatttatgtatattaatgttttgcctgtatttctgtctgtgtgccacatgtatgtctgttgcccacagagtctagaaaagagcactgggtctcctagaactggagtcacagaaggttgtgagccaccatgtcgagtttgaaactaggtgctatagagcagccagtgctcttaaccactgagccagctcccaactgtatgttgaatttgttgacctttatcgtgcaccgaatgtaaatataatggaactctcctgacacttaagttgagggaaaggacaaaaaaaaaaaaaaaaaa

SEQ ID NO: 4 - protein, 317 aa, Mus musculus
MGYDVTRFQGDVDEDLICPICSGVLEEPVQAPHCEHAFCNACITQWFSQQQTCPVDRSVVTVAHLRPVPRIMRNMLSKLQIACDNAVFGCSAVVRLDNLMSHLSDCEHNPKRPVTCEQGCGLEMPKDELPNHNCIKHLRSVVQQQQSRIAELEKTSAEHKHQLAEQKRDIQLLKAYMRAIRSVNPNLQNLEETIEYNEILEWVNSLQPARVTRWGGMISTPDAVLQAVIKRSLVESGCPASIVNELIENAHERSWPQGLATLETRQMNRRYYENYVAKRIPGKQAVVVMACENQHMGDDMVQEPGLVMIFAHGVEEI

(19) United States
(12) Patent Application Publication
(10) Pub. No.: US 2023/0241250 A1
(43) Pub. Date: Aug. 3, 2023

(54) Methods and Compositions to Target and Treat Macrophages

(71) Applicant: Massachusetts Institute of Technology, Cambridge, MA (US)

(72) Inventors: Elazer R. Edelman, Brookline, MA (US); Pedro Melgar Lesmes, Barcelona (ES)

(21) Appl. No.: 18/163,253

(22) Filed: Feb. 1, 2023

Related U.S. Application Data
(60) Provisional application No. 63/267,428, filed on Feb. 1, 2022.

(51) Int. Cl. A61K 48/00; A61K 38/45; A61P 1/16

(52) U.S. Cl. CPC A61K 48/0058; A61K 38/45; A61P 1/16; A61K 48/0033; A61K 48/0041

(57) ABSTRACT

Provided herein are, in various embodiments, methods and compositions of inducing M2-like macrophage morphology. In certain embodiments, a composition comprising a polynucleotide encoding a ring finger protein 41 (RNF41) is contemplated. The disclosure also provides a method of preventing, treating, managing, and/or ameliorating tissue damage in a subject in need thereof. In some embodiments, the subject has chronic liver disease, chronic liver inflammation, chronic hepatic fibrosis, cirrhosis, or a combination thereof. In still further embodiments, the subject has undergone liver resection or liver transplantation.

### RELATED APPLICATION

This application claims the benefit of U.S. Provisional Application No. 63/267,428, filed on Feb. 1, 2022. The entire teachings of the above application are incorporated herein by reference.

### INCORPORATION BY REFERENCE OF MATERIAL IN XML

This application incorporates by reference the Sequence Listing contained in the following eXtensible Markup Language (XML) file being submitted concurrently herewith:

a) File name: 00502367001.xml; created Feb. 1, 2023, 14,899 Bytes in size.

### BACKGROUND

Chronic liver disease accounts for nearly two million deaths per year worldwide. Cirrhosis is within the top 20 causes of disability-adjusted life years (DALY) and years of life lost. No curative solutions exist for cirrhosis except for organ transplantation, which requires significant surgery and lifelong immunosuppression. Yet only 50% of eligible patients receive a liver transplant, which translates into a shortage of about 13,000 donors per year.

Accordingly, new strategies to treat liver disease and stimulate hepatic regeneration are needed.

### SUMMARY

In one aspect, the present disclosure provides a method of inducing M2-like macrophage morphology and/or phenotype in a macrophage, comprising contacting a macrophage with a composition comprising a polynucleotide encoding a ring finger protein 41 (RNF41) (i.e., a RNF41 encoding sequence), under conditions whereby the composition is phagocytized by the macrophage and RNF41 is expressed and/or RNF41 levels are increased. In certain embodiments, the macrophage has elevated anti-inflammatory factors, elevated anti-fibrotic factors, elevated pro-regenerative factors, or a combination thereof.

In certain aspects, the present disclosure provides a method of inducing M2-like macrophage phenotype in a macrophage, comprising contacting a macrophage with a composition, wherein the composition comprises a polynucleotide encoding a ring finger protein 41 (RNF41), under conditions whereby the composition enters the macrophage and RNF41 is expressed and/or RNF41 levels are increased.

In another aspect, the present disclosure provides a method of preventing, treating, managing, and/or ameliorating tissue damage in a subject in need thereof, comprising administering to the subject an effective amount of a composition comprising a polynucleotide encoding ring finger protein 41 (RNF41). In some embodiments, the composition increases RNF41 levels. In some embodiments, the increased RNF41 levels are in macrophages. In some embodiments, the subject has, or is predisposed to have, tissue injury. In still further embodiments, the subject has chronic liver disease, chronic liver inflammation, chronic hepatic fibrosis, cirrhosis, or a combination thereof. In still further embodiments, the subject has undergone liver resection or liver transplantation.

In yet another aspect, the disclosure provides a method of promoting hepatic regeneration in a subject in need thereof, comprising administering to the subject an effective amount of a composition comprising a polynucleotide encoding ring finger protein 41 (RNF41), wherein the subject has undergone a hepatectomy.

In a further aspect, the present disclosure provides a composition comprising a polynucleotide encoding a ring finger protein 41 (RNF41), wherein the polynucleotide encoding RNF41 is a plasmid, and wherein the plasmid is operably linked to a graphite nanoparticle.

In yet another aspect, the present disclosure provides a pharmaceutical composition, comprising any one of the agents described herein, and one or more pharmaceutically acceptable excipients, diluents, or carriers.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1G show that macrophage RNF41 and its stabilizer USP8 are downregulated in cirrhotic livers in part due to chronic inflammation. FIG. 1A shows RNF41 expression in CD11b+-macrophages isolated from the livers of patients with liver cirrhosis (N=6) and healthy subjects (N=4). FIG. 1B shows USP8 expression in CD11b+-macrophages isolated from the livers of patients with liver cirrhosis and healthy subjects. FIG. 1C shows RNF41 expression in CD11b+-macrophages isolated from the livers of healthy and cirrhotic mice (N=6). FIG. 1D shows USP8 expression in CD11b+-macrophages isolated from the liver of healthy and cirrhotic mice (N=6). FIG. 1E shows RNF41 expression in RAW 264.7 macrophages stimulated with TNF-α for 7 days. FIG. 1F shows USP8 expression in RAW 264.7 macrophages stimulated with TNF-α for 7 days. The experiments of FIG. 1E and FIG. 1F were performed in triplicate. For FIG. 1E and FIG. 1F, * indicates P≤0.05, ** indicates P≤0.01, *** indicates P≤0.001, **** indicates P≤0.0001 vs. day 0 using a Student's t-test. Data is shown as mean±S.D. FIG. 1G shows Western-Blot analysis of phospho-Akt, total Akt, phospho-Erk, total Erk and β-actin in RAW 264.7 macrophages stimulated with TNF-α for 7 days.

FIGS. 2A-2I show that dendrimer-graphite nanoparticles are macrophage-selective plasmid-delivery vectors for effective gene therapy. FIG. 2A shows the structure of graphite nanoparticles linked to dendrimer and plasmid DNA. FIG. 2B-1 shows TEM images of graphite nanoparticles. FIG. 2B-2 shows the graph of the particle size (nm) of graphite nanoparticles measured using the TEM images of FIG. 2B-1. FIG. 2C-1 shows TEM images of dendrimer-graphite nanoparticles. FIG. 2C-2 shows the graph of the particle size (nm) of dendrimer-graphite nanoparticles measured using the TEM images of FIG. 2C-1. FIG. 2D shows Z-average size and polydispersity index (PDI) of graphite nanoparticles measured using dynamic light scattering. FIG. 2E shows Z-average size and PDI of dendrimer-graphite nanoparticles measured using dynamic light scattering. FIG. 2F shows osmolality and zeta potential of every graphite nanoparticle composite. FIG. 2G shows RAW 264.7 macrophage intracellular distribution of FITC-dendrimer-graphite nanoparticles. FIG. 2H shows cell morphology of RAW 264.7 macrophage treated with TNF-α and dendrimer-graphite nanoparticles linked to plasmids (pSCR or pRNF41), and observed using light microscopy. FIG. 2I shows immunofluorescence staining for CD206 in RAW 264.7 macrophages treated with TNF-α and dendrimer-graphite nanoparticles linked to plasmids (pSCR or pRNF41).

FIGS. 3A-3F show dendrimer-graphite nanoparticles are efficiently and selectively transfecting a RNF41 plasmid in macrophages recruited to the mouse cirrhotic liver. FIG. 3A shows immunofluorescence staining for Ly6C and simultaneous detection of EGFP fluorescence in the liver of cirrhotic mice treated with dendrimer-graphite nanoparticles linked to pSCR. FIG. 3B shows immunofluorescence staining for CD206 and simultaneous detection of EGFP fluorescence in the liver of cirrhotic mice treated with dendrimer-graphite nanoparticles linked to pSCR. FIG. 3C shows immunofluorescence staining for CD206 and simultaneous detection of EGFP fluorescence in the liver of cirrhotic mice treated with dendrimer-graphite nanoparticles linked to pRNF41. FIG. 3D shows detection of EGFP fluorescence in the kidney of cirrhotic animals treated with dendrimer-graphite nanoparticles linked to pSCR. FIG. 3E shows detection of EGFP fluorescence in the spleen of cirrhotic animals treated with dendrimer-graphite nanoparticles linked to pSCR. FIG. 3F shows detection of EGFP fluorescence in the lungs of cirrhotic mice treated with dendrimer-graphite nanoparticles linked to pSCR. All plasmids constitutively expressed EGFP under the control of a CMV promoter. Scale bar: 200 μm.

FIGS. 4A-4H show RNF41 restoration in macrophages located into the cirrhotic liver orchestrates fibrosis and inflammation regression, and recovery of hepatic function. FIG. 4A shows RNF41 expression in healthy and cirrhotic mice treated with dendrimer-graphite nanoparticles linked to plasmids (scrambled, pSCR or expressing RNF41, pRNF41). FIG. 4B shows macroscopic aspect of cirrhotic liver changing from micronodular cirrhotic liver to an apparently non-cirrhotic liver when treated with pRNF41-DGNP. FIG. 4C-1 shows Sirius Red staining of fibrosis area in the liver of cirrhotic mice treated with dendrimer-graphite nanoparticles linked to plasmids (pSCR or pRNF41). FIG. 4C-2 shows quantification of FIG. 4C-1 images. FIG. 4D shows serum parameters of liver function in cirrhotic mice treated with dendrimer-graphite nanoparticles linked to plasmids (pSCR or pRNF41). FIG. 4E-1 shows proliferating cell nuclear antigen (PCNA) immunofluorescence staining in the liver of cirrhotic mice treated with dendrimer-graphite nanoparticles linked to plasmids (pSCR or pRNF41). FIG. 4E-2 shows quantification of FIG. 4E-1. FIG. 4F shows hepatic expression of hepatocyte growth factor (HGF) and insulin-like growth factor 1 (IGF-1) in cirrhotic mice treated with dendrimer-graphite nanoparticles linked to plasmids (pSCR or pRNF41). FIG. 4G shows cell proliferation in isolated mouse hepatocytes treated for 24 h with conditioned media from RAW 264.7 cultures treated with FBS, TNF-α, dendrimer-graphite nanoparticles linked to plasmids (pSCR or pRNF41, or pshRNF41) or IGF-1 antibody for 3 days. FIG. 4H shows expression of M1 and M2-related genes in the liver of cirrhotic mice treated with dendrimer-graphite nanoparticles linked to plasmids (pSCR or pRNF41). N=6 per group of animals. ** indicates P≤0.01, *** indicates P≤0.001, **** indicates P≤0.0001 using a Student's t-test. For g, *** indicates P≤0.001 using one-way ANOVA with the posthoc Newman-Keuls test. Data is shown as mean±S.D.

FIGS. 5A-5I show depletion of macrophage RNF41 worsens fibrosis, inflammation, and hepatic damage in cirrhotic mice. FIG. 5A shows gene expression of RNF41 in the liver of cirrhotic mice treated with dendrimer-graphite nanoparticles linked to plasmids (pshSCR or pshRNF41). FIG. 5B shows survival rate of cirrhotic mice treated with dendrimer-graphite nanoparticles linked to plasmids (pshSCR or pshRNF41). FIG. 5C-1 shows Sirius Red staining in cirrhotic mice treated with dendrimer-graphite nanoparticles linked to plasmids (pshSCR or pshRNF41). FIG. 5C-2 shows quantification of FIG. 5C-1. FIG. 5D shows expression of genes related to hepatic stellate cell (HSC) activation in the liver of cirrhotic mice treated with dendrimer-graphite nanoparticles linked to plasmids (pshSCR or pshRNF41). FIG. 5E shows gene expression of pro-fibrogenic agents produced by macrophages in the liver of cirrhotic mice treated with dendrimer-graphite nanoparticles linked to plasmids (pshSCR or pshRNF41). FIG. 5F shows serum parameters of liver function in cirrhotic mice treated with dendrimer-graphite nanoparticles linked to plasmids (pSCR or pRNF41). FIG. 5G shows expression of inflammatory M1 and anti-inflammatory M2 macrophage genes in the liver of cirrhotic mice treated with dendrimer-graphite nanoparticles linked to plasmids (pshSCR or pshRNF41). FIG. 5H-1 shows PCNA immunofluorescence staining in the liver of cirrhotic mice treated with dendrimer-graphite nanoparticles linked to plasmids (pshSCR or pshRNF41). FIG. 5H-2 shows quantification of FIG. 5H-1. FIG. 5I shows hepatic expression of hepatocyte growth factor (HGF) and insulin-like growth factor 1 (IGF-1) in cirrhotic mice treated with dendrimer-graphite nanoparticles linked to plasmids (pSCR or pRNF41). N=6 per group of animals. * indicates P≤0.05, ** indicates P≤0.01, *** indicates P≤0.001, **** indicates P≤0.0001 using a Student's t-test. Data is shown as mean±S.D.

FIGS. 6A-6H show RNF41 induces regeneration after hepatectomy in part via insulin-like growth factor 1 (IGF-1) induction. FIG. 6A shows liver restoration rate in healthy mice undergoing 70% hepatectomy and treated with dendrimer-graphite nanoparticles linked to plasmids (pSCR or pRNF41). FIG. 6B-1 shows PCNA immunofluorescence staining in the liver of healthy mice undergoing 70% hepatectomy and treated with dendrimer-graphite nanoparticles linked to plasmids (pSCR or pRNF41). FIG. 6B-2 shows quantification of FIG. 6B-1. FIG. 6C shows serum parameters of liver function in healthy mice undergoing 70% hepatectomy and treated with dendrimer-graphite nanoparticles linked to plasmids (pSCR or pRNF41). FIG. 6D shows hepatic expression of hepatocyte growth factor (HGF) and IGF-1 in healthy mice undergoing 70% hepatectomy and treated with dendrimer-graphite nanoparticles linked to plasmids (pSCR or pRNF41). FIG. 6E shows liver restoration rate in cirrhotic mice undergoing 40% hepatectomy and treated with dendrimer-graphite nanoparticles linked to plasmids (pSCR or pRNF41). FIG. 6F-1 shows PCNA immunofluorescence staining in the liver of cirrhotic mice undergoing 40% hepatectomy and treated with dendrimer-graphite nanoparticles linked to plasmids (pSCR or pRNF41). FIG. 6F-2 shows quantification of FIG. 6F-1. FIG. 6G shows serum parameters of liver function in cirrhotic mice undergoing 40% hepatectomy and treated with dendrimer-graphite nanoparticles linked to plasmids (pSCR or pRNF41). FIG. 6H shows hepatic expression of HGF and IGF-1 in cirrhotic mice undergoing 40% hepatectomy and treated with dendrimer-graphite nanoparticles linked to plasmids (pSCR or pRNF41). N=6 per group of animals. * indicates P≤0.05, ** indicates P≤0.01, *** indicates P≤0.001, **** indicates P≤0.0001 using a Student's t-test. Data is shown as mean±S.D.

FIG. 7A shows structure of pRNF41 plasmid. FIG. 7B shows the structure of pshRNF41 plasmid.

FIG. 8A-8D show toxicity and uptake of plasmid-dendrimer graphite nanoparticles. FIG. 8A shows HUVEC viability quantified by MTS in the presence of pRNF41-dendrimer-graphite nanoparticles at concentrations from 5 to 500 μg/mL at 24 h. No significant differences (ns) observed using Student's t-test compared to non-treated control. FIG. 8B shows uptake kinetics in RAW 264.7 macrophages of pRNF41-DGNP in the presence or absence of TNF-α. * indicates P≤0.05, ** indicates P≤0.01, *** indicates P≤0.001 **** indicates P≤0.0001 vs. macrophages without TNF-α at the same time point using Student's t-test. FIG. 8C-1 shows uptake percentage in RAW 264.7 macrophages of pRNF41-DGNP at 24 h. FIG. 8C-2 shows quantification of FIG. 8C-1 where *** indicates P≤0.001 using a Student's t-test. FIG. 8D shows plasmid transfection and expression efficiency highlighted by the presence of high levels of RAW 264.7 macrophage intracellular EGFP in most of cells after 3 days of incubation in TNF-α presence. Data is shown as mean±S.D.

FIGS. 9A-9B show functional assay of gelatinase activity in macrophages treated with dendrimer-graphite nanoparticles linked to pRNF41. FIG. 9A shows fluorescence images of RAW 264.7 macrophages seeded on FITC-gelatin coated plates and treated with dendrimer-graphite nanoparticles linked to pRNF41 (pRNF41-DGNP) for 5 days displaying a black halo as a consequence of the collagen digestion and a green nuclear staining as a consequence of the EGFP expression. FIG. 9B shows time-course quantitative analysis of FITC released to the medium in the gelatinase activity assay in the presence or absence of TNF-α or pRNF41-DGNP for 7 days (N=4). **** indicates P≤0.0001 vs. macrophages without TNF-α and with or without pRNF41-DGNP at the same time point using a one-way analysis of variance (ANOVA) one-way analysis of variance (ANOVA) with the posthoc Newman-Keuls test. RFU: relative fluorescence units. Data is shown as mean±S.D.

FIGS. 10A-10C show hepatic stellate cell inactivation by the treatment with pRNF41-dendrimer-graphite nanoparticles in cirrhotic mice. FIG. 10A shows collagen I, FIG. 10B shows α-SMA and FIG. 10C shows TIMP-1 expression in the liver of cirrhotic mice treated with dendrimer-graphite nanoparticles linked to plasmids (pSCR or pRNF41). ** indicates P≤0.01, **** indicates P≤0.0001 using a Student's t-test. Data is shown as mean±S.D.

FIGS. 11A-11C show therapy with pRNF41-dendrimer-graphite reduces the expression of macrophage-derived activators of hepatic stellate cells in cirrhotic mice. FIG. 11A shows OSM, FIG. 11B shows PDGF-BB and FIG. 11C shows TGF-β expression in the liver of cirrhotic mice treated with dendrimer-graphite nanoparticles linked to plasmids (pSCR or pRNF41). ** indicates P≤0.01, *** indicates P≤0.001, **** indicates P≤0.0001 using a Student's t-test. Data is shown as mean±S.D.

FIGS. 12A-12B show the treatment with pRNF41-dendrimer-graphite nanoparticles activates the expression of metalloproteinase 9 and regenerates liver mass in cirrhotic mice. FIG. 12A shows MMP-9 expression in the liver of cirrhotic mice treated with dendrimer-graphite nanoparticles linked to plasmids (pSCR or pRNF41). FIG. 12B shows liver restoration rate in cirrhotic mice treated with dendrimer-graphite nanoparticles linked to plasmids (pSCR or pRNF41). * indicates P≤0.05, ** indicates P≤0.01 using a Student's t-test. Data is shown as mean±S.D.

FIGS. 13A-13C show IGF-1 released by RNF41-activated macrophages reduces pro-fibrogenic activation of LX-2 human hepatic stellate cells. FIG. 13A shows collagen I, FIG. 13B shows α-SMA and FIG. 13C shows TIMP-1 expression in LX-2 cells treated 24 h with conditioned media from RAW 264.7 cultures treated with FBS, TNF-α, dendrimer-graphite nanoparticles linked to plasmids (pSCR or pRNF41, or pshRNF41) or IGF-1 antibody for 3 days. * indicates P≤0.05; ** indicates P≤0.01, **** indicates P≤0.0001 using a one-way analysis of variance (ANOVA) with the posthoc Newman-Keuls test. Experiments were performed in sextuplicate. Data is shown as mean±S.D.

FIGS. 14A-14F show the treatment with pRNF41-dendrimer-graphite nanoparticles activates the expression of the downstream PPAR-γ genes IL-10 and CD36 in the liver. FIG. 14A shows IL-10 and FIG. 14B shows CD36 expression in the liver of cirrhotic mice treated with dendrimer-graphite nanoparticles linked to plasmids (pSCR or pRNF41). FIG. 14C shows IL-10 and FIG. 14D shows CD36 expression in the liver of healthy mice undergoing 70% hepatectomy and treated with dendrimer-graphite nanoparticles linked to plasmids (pSCR or pRNF41). FIG. 14E shows IL-10 and FIG. 14F shows CD36 expression in the liver of cirrhotic mice undergoing 40% hepatectomy and treated with dendrimer-graphite nanoparticles linked to plasmids (pSCR or pRNF41). ** indicates P≤0.01, *** indicates P≤0.001, **** indicates P≤0.001 using a Student's t-test. Data is shown as mean±S.D.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an” and “the” should be understood to include plural reference unless the context clearly indicates otherwise.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the invention, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Hepatic inflammation is a common trigger of chronic liver disease. Namely, macrophage activation is a predictive parameter for survival in cirrhotic patients. Macrophages are cellular regulators involved in all stages of liver disease: from initial tissue injury to chronic inflammation, fibrosis and repair. Resident hepatic macrophages release signals that promote local immune response and limit initial injury through the classic path of inflammatory cell recruitment and subsequent activation of hepatic stellate cells with production of a supporting extracellular matrix (ECM). When injury abates, macrophages remodel the fibrosis primarily by releasing matrix metalloproteinases (MMPs), which promote fibrotic ECM degradation and repair through elaboration of factors that reduce the inflammatory response and boosts hepatic regeneration.

Ring finger protein 41 (RNF41), also known as neuregulin receptor degradation protein-1 (Nrdp1) or fetal liver ring finger (FLRF), is an E3 ubiquitin protein ligase that plays an essential role in the degradation of pro-inflammatory JAK2-associated cytokine receptors, adaptors and kinases. Wauman, J., De Ceuninck, L., Vanderroost, N., Lievens, S. & Tavernier, J. RNF41 (Nrdp1) controls type 1 cytokine receptor degradation and ectodomain shedding. J Cell Sci 124, 921-932 (2011). This ligase inhibits the production of proinflammatory cytokines in Toll-like receptor-triggered macrophages via suppression of MyD88 and NF-κB activation and confers resistance to lipopolysaccharide-induced endotoxin shock. Wang, C. et al. The E3 ubiquitin ligase Nrdp1 ‘preferentially’ promotes TLR-mediated production of type I interferon. Nat Immunol 10, 744-752 (2009). RNF41 also promotes M2 macrophage polarization by ubiquitination and activation of the transcription factor CCAAT/Enhancer-binding Protein β (C/EBPβ) Ye, S. et al. The E3 ubiquitin ligase neuregulin receptor degradation protein 1 (Nrdp1) promotes M2 macrophage polarization by ubiquitinating and activating transcription factor CCAAT/enhancer-binding Protein beta (C/EBPbeta). J Biol Chem 287, 26740-26748 (2012), which has been associated to muscle injury repair. Ruffell, D. et al. A CREB-C/EBPbeta cascade induces M2 macrophage-specific gene expression and promotes muscle injury repair. Proc Natl Acad Sci USA 106, 17475-17480 (2009). The ring finger protein 41 (RNF41) has been linked to negative regulation of pro-inflammatory cytokines and receptors; however, the precise involvement of macrophage RNF41 in liver cirrhosis has been unknown.

Considering the crucial influence that macrophages exert on the modulation of the hepatic cellular response to injury, the present disclosure uses a nanoscale gene therapy delivery system designed to modulate inflammatory macrophages for the harmonization of fibrosis resolution and hepatic regeneration.

This disclosure demonstrates that RNF41 is downregulated by the sustained inflammatory milieu of the cirrhotic liver and that further depletion of RNF41 expression in macrophages from cirrhotic livers accentuated hepatic inflammation and damage with a deep impact in survival. These data show that macrophage RNF41 function is applicable to chronic liver disease and translatable to humans.

Selective macrophage gene therapy using plasmid-dendrimer-graphite nanoparticles restored macrophage RNF41 expression, which resulted in complete resolution of fibrosis in cirrhotic mice and enhanced hepatic regeneration with significant improved liver function. RNF41 acted both as a negative regulator in the macrophage synthesis of inflammatory and pro-fibrogenic cytokines, and a positive regulator of anti-inflammatory, anti-fibrotic and pro-regenerative factors, in part via IGF-1 and peroxisome proliferator-activated receptors gamma (PPAR-γ) induction. These data reveal that sustained inflammatory signals from TNF-α promote down-regulation of macrophage RNF41 and its stabilizer USP8. In contrast, short-term TNF-α exposition induces RNF41 and USP8 expression in macrophages as described using other inflammatory factors.

As disclosed herein, cell culture experiments, gene expression profiling and liver slice staining substantiated that selective gene therapy to stimulate macrophage RNF41 expression in the liver of cirrhotic animals using pRNF41-DGNP promotes the switch of hepatic macrophages from pro-inflammatory M1-like to anti-inflammatory M2-like phenotype. These findings suggest that macrophage RNF41, through multiple intracellular signals, controls the behavior of recruited macrophages in the cirrhotic liver and leads the local immune response towards hepatic fibrosis resolution and regeneration.

### Methods of the Disclosure

In one aspect, the present disclosure provides a method of inducing M2-like macrophage morphology in a macrophage, comprising contacting a macrophage with a composition, wherein the composition comprises a polynucleotide encoding a ring finger protein 41 (RNF41), under conditions whereby the composition enters the macrophage and RNF41 is expressed. In some embodiments, the means of entry is phagocytosis. In other embodiments, entry may be, e.g., through endocytosis and/or facilitated diffusion.

Ring finger protein 41 (RNF41) is an E3 ubiquitin-protein ligase that regulates the degradation of target proteins, also known as neuregulin receptor degradation protein-1 (Nrdp1) or fetal liver ring finger (FLRF) (corresponding to the UniProtKB Ref Q9H4P4; Ensembl Ref. ENSG00000181852; HGNC Ref 18401; NCBI Entrez Gene Ref. 10193).

In some embodiments, the RNF41 is from a human. In other embodiments, the RNF41 is from a mouse. In still further embodiments, the RNF41 polynucleotide is at least about 70% identical to SEQ ID NO:1 or SEQ ID NO:3, for example, has at least about: 75%, 80%, 85%, 90%, 95%, 96%, 97%, 98%, 99% or 100% sequence identity to SEQ ID NO:1 or SEQ ID NO:3. In certain embodiments, the polynucleotide comprises a nucleotide sequence that is about: 70%, 75%, 80%, 85%, 90%, 95%, 96%, 97%, 98%, 99% or 100% identical to SEQ ID NO:1 or SEQ ID NO:3. In some embodiments, the polynucleotide comprises a nucleotide sequence having about 70%, 75%, 80%, 85%, 90%, 95%, 96%, 97%, 98%, 99% or 100% sequence identity to SEQ ID NO:1 or SEQ ID NO:3. In particular embodiments, the polynucleotide comprises a nucleotide sequence having about 70-100% sequence identity to SEQ ID NO:1 or SEQ ID NO:3, for example, about: 75-100%, 75-99%, 80-100%, 80-98%, 85-100%, 85-97%, 90-100%, 90-96%, 95-100%, 96-100%, 97-100%, 98-100% or 99-100%.

As used herein, the term “sequence identity,” refers to the extent to which two nucleotide sequences have the same residues at the same positions when the sequences are aligned to achieve a maximal level of identity, expressed as a percentage. For sequence alignment and comparison, typically one sequence is designated as a reference sequence, to which a test sequences are compared. Sequence identity between reference and test sequences is expressed as a percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide residue at 70% of the same positions over the entire length of the reference sequence.

Alignment of sequences for comparison to achieve maximal levels of identity can be readily performed by a person of ordinary skill in the art using an appropriate alignment method or algorithm. In some instances, alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

In some embodiments, the RNF41 is at least about 70% identical to SEQ ID NO:2 or SEQ ID NO:4, for example, at least about: 75%, 80%, 85%, 90%, 95%, 96%, 97%, 98%, 99% or 100% sequence identity to SEQ ID NO:2 or SEQ ID NO:4. In certain embodiments, the polynucleotide is about: 70%, 75%, 80%, 85%, 90%, 95%, 96%, 97%, 98%, 99% or 100% identical to SEQ ID NO:2 or SEQ ID NO:4. In some embodiments, the polynucleotide is about 70%, 75%, 80%, 85%, 90%, 95%, 96%, 97%, 98%, 99% or 100% sequence identity to SEQ ID NO:2 or SEQ ID NO:4. In particular embodiments, the polynucleotide has about 70-100% sequence identity to SEQ ID NO:2 or SEQ ID NO:4, for example, about: 75-100%, 75-99%, 80-100%, 80-98%, 85-100%, 85-97%, 90-100%, 90-96%, 95-100%, 96-100%, 97-100%, 98-100% or 99-100%.

In some embodiments, the M2-like macrophage morphology consists of elevated expression of mannose receptor CD206, elevated production of matrix metalloproteinases, elevated collagenase activity, or a combination thereof.

### Methods of Treatment

In some embodiments, the disclosure provides for a method of treating tissue damage in a subject in need thereof, the method comprising administering to the subject an effective amount of a composition comprising a polynucleotide encoding ring finger protein 41 (RNF41). In still other embodiments, the composition is phagocytized by a macrophage of the subject and the macrophage expresses RNF41. In some embodiments, the composition increases RNF41 levels. In some embodiments, the increased RNF41 levels are in macrophages.

As used herein, “treat,” “treating,” or “treatment” means inhibiting or relieving a disease or disorder. For example, treatment can include a postponement of development of the symptoms associated with a disease or disorder, and/or a reduction in the severity of such symptoms that will, or are expected, to develop with said disease. The terms include ameliorating or managing existing symptoms, preventing additional symptoms, and ameliorating or preventing the underlying causes of such symptoms. Thus, the terms denote that a beneficial result is being conferred on at least some of the mammals (e.g., humans) being treated. Many medical treatments are effective for some, but not all, subjects that undergo the treatment.

As used herein, “subject” or “patient” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some embodiments, a subject is a mammal (e.g., a non-human mammal). In some embodiments, a subject is a human.

As used herein, the term “effective amount” means an amount of a composition comprising a polynucleotide encoding ring finger protein 41 (RNF41), that when administered alone or in combination with an additional therapeutic agent to a cell, tissue, or subject, is effective to achieve the desired therapeutic or prophylactic effect under the conditions of administration. In certain embodiments, the disclosure provides for the use of an anti-inflammatory as an additional therapeutic agent. In still other embodiments, the additional therapeutic agent is a pro-inflammatory agent. As an example, an effective amount is one that would be sufficient to diminish tissue damage to bring about effectiveness of a therapy. The effectiveness of a therapy (e.g., elevated expression of mannose receptor CD206, elevated production of matrix metalloproteinases, elevated collagenase activity) can be determined by suitable methods known in the art.

“Administering” or “administration” as used herein, refers to taking steps to deliver an agent to a subject, such as a mammal, in need thereof. Administering can be performed, for example, once, a plurality of times, and/or over one or more extended periods. Administration includes both direct administration, including self-administration, and indirect administration, including the act of prescribing a drug or directing a subject to consume an agent. For example, as used herein, one (e.g., a physician) who instructs a subject (e.g., a patient) to self-administer an agent (e.g., a drug), or to have the agent administered by another and/or who provides a patient with a prescription for a drug is administering the agent to the subject.

In some embodiments, the macrophage has elevated anti-inflammatory factors, elevated anti-fibrotic factors, elevated pro-regenerative factors, or a combination thereof. In some embodiments, the elevated factors are significantly elevated. In other embodiments, the factors include Interleukin 10 (IL-10), Interleukin 4 (IL-4), MRC1, RETN1A, CD36, or a combination thereof. In still other embodiments, the macrophage exhibits increased IL-10 acting on other immune cells and/or arginase reducing nitric oxide release from macrophages.

As used herein, “exosome” means a membrane-bounded sub-cellular structure which may comprise proteins, messenger ribonucleic acids, and other biologically active substances. In certain embodiments, the exosome is that of a macrophage. In some embodiments, the macrophage exosome is determined in a tissue sample, cell, or serum sample. In some embodiments, the proteins, messenger ribonucleic acids, and other biologically active substances may be freely released.

In still further embodiments, the macrophage is in a subject, and the method comprises administering to the subject an effective amount of the composition, or a pharmaceutically acceptable salt thereof.

The term “pharmaceutically acceptable salts” embraces salts commonly used to form alkali metal salts and to form addition salts of free acids or free bases. The nature of the salt is not critical, provided that it is pharmaceutically acceptable.

Suitable pharmaceutically acceptable acid addition salts may be prepared from an inorganic acid or an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Pharmaceutically acceptable acidic/anionic salts also include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphosphate, polygalacturonate, salicylate, stearate, subacetate, succinate, sulfate, hydrogensulfate, tannate, tartrate, teoclate, tosylate, and triethiodide salts.

Suitable pharmaceutically acceptable base addition salts include, but are not limited to, metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine. Pharmaceutically acceptable basic/cationic salts also include, the diethanolamine, ammonium, ethanolamine, piperazine and triethanolamine salts.

All of these salts may be prepared by conventional means by treating, for example, a composition described herein with an appropriate acid or base.

In some embodiments, the subject has, or is predisposed to have, tissue injury. In further embodiments, the tissue is liver tissue, muscle tissue, lung tissue, spleen tissue, kidney tissue, a tissue of the mononuclear phagocyte system.

As used herein, the mononuclear phagocyte system is also known as the reticuloendothelial system or macrophage system and forms a part of the immune system that consists of the phagocytic cells derived from precursor cells in the bone marrow located in reticular connective tissue. These cells comprise mononuclear phagocytic cells and tissue macrophages or histiocytes. The main tissue macrophages of this system are located in the lymph nodes, the liver, and the spleen, but they can be also found in other tissues.

In certain embodiments, the disclosure provides for a method wherein the subject has chronic liver disease, liver failure, chronic liver inflammation, chronic hepatic fibrosis, cirrhosis, or a combination thereof. In still other embodiments, the present disclosure provides for a method wherein the subject has undergone a partial or complete hepatectomy, liver transplantation, or a combination thereof.

The term “liver disease” as used herein refers to a hepatic disorder. Generally, a liver disease may be caused by any condition that results in the disturbance of the morphological and/or functional integrity of a body's liver. The etiology and treatment of liver diseases are described, e.g., in Oxford Textbook of Medicine (Warrell, Oxford Textbook of Medicine, David A. Warrell, Timothy M. Cox, John D. Firth, Oxford University Press, USA; Fifth edition (Jul. 22, 2010)).

The term “hepatectomy” as used herein refers to hepatic or liver resection. An important decision in any liver resection is choosing the amount of parenchyma to be removed. Accordingly, as used herein, a hepatectomy may be a partial hepatectomy (e.g., removal of a liver segment or a liver lobe) or total hepatectomy (e.g., complete organ removal).

As used herein “transplanted liver” refers a liver transplanted into a subject and also includes the so-called “partial liver transplant” which corresponds to a graft consisting of the part of the liver of a donor. Liver transplantation also refers to injection of hepatocytes (genetically modified or stimulated to proliferate or differentiate) into the portal vein. Portal vein embolization (PVE) can be used to increase future remnant liver volume in patients scheduled for major liver resection.

In some embodiments, the method of the present disclosure comprises reducing tissue inflammation and/or tissue fibrosis. As described herein, in certain embodiments fibrosis is about 85% decreased.

As used herein, the term “reducing” or “reduce” refers to modulation that decreases the amount or the activity of a given material as dictated by context, relative to a reference (e.g., the level prior to or in an absence of modulation by the agent). In some embodiments, the agent (e.g., composition) decreases tissue inflammation and/or tissue fibrosis, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In certain embodiments, the agent (e.g., composition) decreases tissue inflammation and/or tissue fibrosis, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In particular embodiments, the agent (e.g., composition) decreases tissue inflammation and/or tissue fibrosis, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference.

In still further embodiments, the method comprises inducing tissue repair. In yet further embodiments, the method comprises a combination of reducing tissue inflammation, reducing tissue fibrosis, and inducing tissue repair. In some embodiments, hepatic fibrosis is reduced.

As used herein, the term “inducing” or “induces” refers to modulation that increases the amount or the activity of a given material as dictated by context, relative to a reference (e.g., the level prior to or in an absence of modulation by the agent). In some embodiments, the agent (e.g., composition) increases tissue repair, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95%, 98%, 99%, 100%, 105%, 110%, 120%, or 125% relative to the reference. In certain embodiments, the agent (e.g., composition) increases tissue repair, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In particular embodiments, the agent (e.g., composition) increases tissue repair, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference.

In still further embodiments, the methods and compositions described herein provide for hepatic regeneration stimulation. The phrases “accelerating hepatic regeneration,” “promoting hepatic regeneration,” “boosting hepatic regeneration,” “hepatic regeneration stimulation,” and “increasing hepatic regeneration,” as used herein, are demonstrated by a shorter period needed to reach the final liver mass or increased final liver mass (e.g., as determined by computed tomography), or both, to an increase in the final mass and the rate of reaching that mass, as compared to an untreated control.

The present disclosure, in certain embodiments, provides for a method of promoting hepatic regeneration in a subject in need thereof, the method comprising administering to the subject an effective amount of a composition comprising a polynucleotide encoding ring finger protein 41 (RNF41), wherein the subject has undergone a hepatectomy.

### Polynucleotides and Compositions

In some embodiments, the polynucleotide encoding RNF41 is a plasmid. In some embodiments, the plasmid comprises a promoter. In still further embodiments, the promoter is a selective promoter. In some embodiments of the disclosure, the selective promoter is CD11b.

In certain embodiments, the disclosure provides for a plasmid that is operably linked to a graphite nanoparticle. In some embodiments, the plasmid is operably linked to the graphite nanoparticle by at least one dendrimer, such as a polyamidoamine (PAMAM) generation 5 dendrimer. In some embodiments, the disclosure provides for graphene nanoparticles/dendrimers. In some embodiments, the graphene nanoparticles are those described in Melgar-Lesmes, Pedro, et al. “Graphene-Dendrimer Nanostars for Targeted Macrophage Overexpression of Metalloproteinase 9 and Hepatic Fibrosis Precision Therapy.” Nano letters 18(9) (2018): 5839-5845 (the contents of which are incorporated herein by reference). In further embodiments, the zeta potential of the disclosure is negative. In still further embodiments, the zeta potential is about −31.52 mV.

In still further embodiments, the method of the disclosure provides for a composition at a physiological osmolality. In certain embodiments, physiological osmolality is about 300 mOsmol/Kg.

In some embodiments, the disclosure provides for a composition that is a pharmaceutically acceptable composition.

As used herein, the term “pharmaceutically acceptable” refers to species which are, within the scope of sound medical judgment, suitable for use without undue toxicity, irritation, allergic response and the like, and are commensurate with a reasonable benefit/risk ratio. For example, a substance is pharmaceutically acceptable when it is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio.

A desired dose may conveniently be administered in a single dose, for example, such that the agent is administered once per day, or as multiple doses administered at appropriate intervals, for example, such that the agent is administered 2, 3, 4, 5, 6 or more times per day. The daily dose can be divided, especially when relatively large amounts are administered, or as deemed appropriate, into several, for example 2, 3, 4, 5, 6 or more, administrations. In some embodiments, the compositions will be administered from about 1 to about 6 (e.g., 1, 2, 3, 4, 5 or 6) times per day or, alternatively, as an infusion (e.g., a continuous infusion). In some embodiments, compositions are administered one, twice, or three times a week, or every two weeks, every three weeks or every four weeks. In some embodiments, the dose is in the range of 1 μg/kg up to tens of mg/kg. In some embodiments, the dose is a dose disclosed herein.

Determining the dosage and route of administration for a particular agent, patient and disease or condition is well within the abilities of one of skill in the art. Preferably, the dosage does not cause or produces minimal adverse side effects.

Doses lower or higher than those recited above may be required. Specific dosage and treatment regimens for any particular subject will depend upon a variety of factors, for example, the activity of the specific agent employed, the age, body weight, general health status, sex, diet, time of administration, rate of excretion, drug combination, the severity and course of the disease, condition or symptoms, the subject's disposition to the disease, condition or symptoms, the judgment of the treating physician and the severity of the particular disease being treated. The amount of an agent in a composition will also depend upon the particular agent in the composition.

In some embodiments, the concentration of one or more active agents provided in a composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, or 0.01% w/w, w/v or v/v; and/or greater than 90%, 80%, 70%, 60%, 50%, 40%, 30%, 20%, 10%, 5%, 1%, 0.5%, 0.4%, 0.3%, 0.2%, 0.1%, or 0.01% w/w, w/v, or v/v.

In some embodiments, the concentration of one or more active agents provided in a composition is in the range from about 0.01% to about 50%, about 0.01% to about 40%, about 0.01% to about 30%, about 0.05% to about 25%, about 0.1% to about 20%, about 0.15% to about 15%, or about 1% to about 10% w/w, w/v or v/v. In some embodiments, the concentration of one or more active agents provided in a composition is in the range from about 0.001% to about 10%, about 0.01% to about 5%, about 0.05% to about 2.5%, or about 0.1% to about 1% w/w, w/v or v/v.

In embodiments of the present disclosure, the compositions described herein and/or produced using the vectors and/or methods described herein, may be provided in compositions, e.g., pharmaceutical compositions.

Therefore, in some embodiments, the invention also relates to compositions, e.g., compositions comprising a polypeptide or other active agent disclosed herein and a pharmaceutically acceptable carrier. In one aspect, the present disclosure provides pharmaceutical compositions comprising an effective amount of a polynucleotide or other active agent described herein and a pharmaceutically acceptable excipient. Pharmaceutical compositions of the present disclosure may comprise a polynucleotide as described herein, in combination with one or more pharmaceutically or physiologically acceptable carriers, excipients or diluents. In some embodiments, pharmaceutical compositions of the present disclosure may comprise one or more pharmaceutically or physiologically acceptable carriers, excipients or diluents.

In some embodiments, a pharmaceutically acceptable carrier can be an ingredient in a pharmaceutical composition, other than an active ingredient, which is nontoxic to the subject.

A pharmaceutically acceptable carrier can include, but is not limited to, a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, saccharides, antioxidants, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation.

In certain embodiments, compositions of the present disclosure can be formulated for a variety of means of parenteral or non-parenteral administration. In one embodiment, the compositions can be formulated for infusion or intravenous administration. Compositions disclosed herein can be provided, for example, as sterile liquid preparations, e.g., isotonic aqueous solutions, emulsions, suspensions, dispersions, or viscous compositions, which may be buffered to a desirable pH. Formulations suitable for oral administration can include liquid solutions, capsules, sachets, tablets, lozenges, and troches, powders liquid suspensions in an appropriate liquid and emulsions.

Stimulation with TNF-α

In certain embodiments, the disclosure provides a method wherein, prior to contacting the macrophage with a composition, the macrophage has been stimulated with tumor necrosis factor alpha (TNF-α).

In certain embodiments, TNF-α stimulation is resultant from cardiovascular disease, diabetes, an auto-immune disease, allergic asthma, inflammatory bowel disease, chronic hepatic and/or renal disease, malignancy, Alzheimer's disease, or a combination thereof.

In still other embodiments, TNF-α stimulation occurs by exogenous treatment with TNF-α.

### ADDITION ENUMERATED EMBODIMENTS

Embodiment 1: A method of inducing M2-like macrophage morphology/phenotype in a macrophage, comprising contacting a macrophage with a composition, wherein the composition comprises a polynucleotide encoding a ring finger protein 41 (RNF41), under conditions whereby the composition enters the macrophage and RNF41 is expressed and/or RNF41 levels are increased.

Embodiment 2: The method of Embodiment 1, wherein the M2-like macrophage morphology/phenotype consists of elevated expression of mannose receptor CD206, elevated production of matrix metalloproteinases, elevated collagenase activity, or a combination thereof.

Embodiment 3: A method of treating tissue damage in a subject in need thereof, the method comprising administering to the subject an effective amount of a composition comprising a polynucleotide encoding ring finger protein 41 (RNF41).

Embodiment 4: The method of Embodiment 3, wherein the composition is phagocytized by a macrophage of the subject and wherein the macrophage expresses RNF41.

Embodiment 5: The method of Embodiment 1, 2, or 4, wherein the macrophage has elevated anti-inflammatory factors, elevated anti-fibrotic factors, elevated pro-regenerative factors, or a combination thereof.

Embodiment 6: The method of any one of Embodiment 1, 2, 4, or 5, wherein the macrophage is in a subject, and the method comprises administering to the subject an effective amount of the composition, or a pharmaceutically acceptable salt thereof.

Embodiment 7: The method of Embodiment 6, wherein the subject has, or is predisposed to have, tissue injury.

Embodiment 8: The method of Embodiment 7, wherein the tissue is liver tissue, muscle tissue, lung tissue, spleen tissue, kidney tissue, a tissue of the mononuclear phagocyte system, or a combination thereof.

Embodiment 9: The method of any one of Embodiment 6-8, wherein the subject has chronic liver disease, liver failure, chronic liver inflammation, chronic hepatic fibrosis, cirrhosis, or a combination thereof.

Embodiment 10: The method of any one of Embodiment 6-8, wherein the subject has undergone a partial or complete hepatectomy, liver transplantation, or a combination thereof.

Embodiment 11: The method of any one of Embodiment 3, 7 or 8, wherein tissue inflammation is reduced, tissue fibrosis is reduced, tissue repair is induced, or a combination thereof.

Embodiment 12: The method of any one of Embodiments 9-11, wherein hepatic fibrosis is reduced.

Embodiment 13: The method of Embodiment 12, wherein the hepatic fibrosis is about 85% reduced.

Embodiment 14: The method of any one of Embodiment 9-13, wherein hepatic regeneration is stimulated.

Embodiment 15: A method of promoting hepatic regeneration in a subject in need thereof, the method comprising administering to the subject an effective amount of a composition comprising a polynucleotide encoding ring finger protein 41 (RNF41), wherein the subject has undergone a hepatectomy.

Embodiment 16: The method of any one of Embodiment 1-15, wherein the polynucleotide encoding RNF41 is a plasmid.

Embodiment 17: The method of Embodiment 16, wherein the plasmid further comprises a promoter.

Embodiment 18: The method of Embodiment 17, wherein the promoter is a selective promoter.

Embodiment 19: The method of Embodiment 18, wherein the selective promoter is CD11b.

Embodiment 20: The method of any one of Embodiment 16-19, wherein the plasmid is operably linked to a graphite nanoparticle.

Embodiment 21: The method of Embodiment 20, wherein the plasmid is operably linked to the graphite nanoparticle by at least one dendrimer.

Embodiment 22: The method of Embodiment 20 or 21, wherein the plasmid is operably linked to the graphite nanoparticle by at least one PAMAM generation 5 dendrimer.

Embodiment 23: The method of any one of Embodiment 20-22, wherein the zeta potential is negative.

Embodiment 24: The method of any one of Embodiment 20-23, wherein the zeta potential is about −31.52 mV.

Embodiment 25: The method of any one of Embodiment 15-24, wherein the composition is at a physiological osmolality.

Embodiment 26: The method of Embodiment 25, wherein the physiological osmolality is about 300 mOsmol/Kg.

Embodiment 27: The method of any one of Embodiment 1-26, wherein, prior to contacting the macrophage with the composition, the macrophage has been stimulated with tumor necrosis factor alpha (TNF-α).

Embodiment 28: The method of Embodiment 27, wherein the stimulation with TNF-α is a result of cardiovascular disease, diabetes, an auto-immune disease, allergic asthma, inflammatory bowel disease, chronic hepatic and/or renal disease, malignancy, Alzheimer's disease, or a combination thereof.

Embodiment 29: The method of Embodiment 28, wherein stimulation with TNF-α is a result of exogenous treatment with TNF-α, lipopolysaccharide (LPS) stimulation, or a combination thereof.

Embodiment 30: The method of Embodiment 1, wherein the macrophage is a tumor-associated macrophage.

Embodiment 31: The method of Embodiment 3, wherein the subject has monocyte-related leukemia, rheumatoid arthritis, Alzheimer's disease, type 2 diabetes, asthma, heart disease, obesity, cancer, inflammatory bowel disease, or a combination thereof.

Embodiment 32: The method of Embodiment 3, further comprising administering an additional therapeutic agent.

Embodiment 33: A composition comprising a polynucleotide encoding a ring finger protein 41 (RNF41), wherein the polynucleotide encoding RNF41 is a plasmid, and wherein the plasmid is operably linked to a graphite nanoparticle.

Embodiment 34: The composition of Embodiment 33, wherein the plasmid further comprises a promoter.

Embodiment 35: The composition of Embodiment 34, wherein the promoter is a selective promoter.

Embodiment 36: The composition of Embodiment 35, wherein the selective promoter is CD11b.

Embodiment 37: The composition of any one of Embodiment 33-36, wherein the plasmid is operably linked to the graphite nanoparticle by at least one dendrimer.

Embodiment 38: The composition of any one of Embodiment 33-37, wherein the plasmid is operably linked to the graphite nanoparticle by at least one PAMAM generation 5 dendrimer.

Embodiment 39: The composition of any one of Embodiment 33-38, wherein the composition is formulated to be a pharmaceutically acceptable composition.

Embodiment 40: A method of making a ring finger protein 41 (RNF41) nanosystem, comprising

oxidizing a graphite nanoparticle to obtain a graphite oxide nanoparticle with surface carboxylic acid groups (cGNP);

chemically attaching dendrimers to the cGNP to obtain a dendrimer-GNP (DGNP); and

adding an RNF41 expression plasmid to the DGNP.

### EXEMPLIFICATION

### Example 1. Methods

No statistical methods were used to predetermine sample size. The experiments were randomized. The investigators were blinded to allocation during experiments and outcome assessment unless otherwise stated in the methods.

Patients. All protocols conformed to the ethical guidelines of the 1975 Declaration of Helsinki and were approved by the Ethics Committee of the Hospital Clinic of Barcelona. All the patients included in this study provided written and signed informed consent. Human normal liver samples were obtained from small biopsies from donor liver lobules during transplantation (n=4). All subjects had normal hepatic histology and no declared acute or chronic diseases. Human cirrhotic liver samples were obtained from liver explants of patients with end-stage cirrhosis caused by non-viral liver disease (n=6) undergoing liver transplantation.

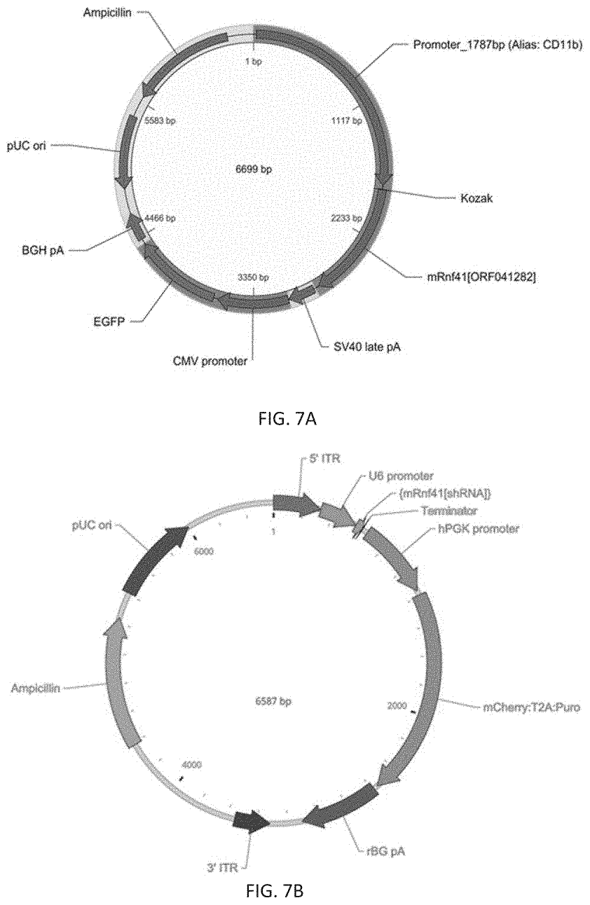

Synthesis and functionalization of dendrimer-graphite nanoparticles. Carbon graphite nanoparticles were supplied by Graphene Supermarket (Reading, Mass., USA). Generation 5 PAMAM dendrimer was purchased from Dendritech Inc. (Midland, Mich.). Scrambled and RNF41 or shRNF41 expression plasmids (FIG. 7) were obtained from Cyagen Biosciences (Guangzhou, China). One Shot® Top 10 Chemically Competent E. coli and Qiagen® Endofree Plasmid Maxi Kit, used for transformation, amplification, and purification of ultrapure, transfection-grade plasmid DNA, were purchased from Thermo Fisher Scientific Inc. (Waltham, Mass., USA) and Qiagen Inc. (Chatsworth, Calif., USA), respectively. Luria broth (LB broth) and LB agar ampicillin-100 plates for bacterial selection were obtained from Sigma (St. Louis, Mo.). Deionized water was obtained from a Milli-Q water purification system (Millipore, Molsheim, France). Graphite nanoparticles were dispersed in deionized water (500 μg/mL) and oxidized using a modified Hofmann method (68% HNO₃/96% H₂SO₄ at 3:1 ratio in the presence of 70 μM KClO₃) in continuous magnetic stirring for 96 h. Dispersion was then neutralized with NaOH until pH=7 and centrifuged at 21.000 g for 30 min. Supernatant with small graphene oxide sheets was discarded, and graphite oxide nanoparticles washed four times with distilled water and centrifuged at 21,000 g for 30 min. Oxidized graphite nanoparticles were separated by incubating the dispersion in an ultrasound bath (Selecta, Barcelona, Spain), at a frequency of 50 kHz and potency 360 W for 15 min. Afterwards 100 μL of graphite nanoparticles were mixed with 900 μL of 1 mg/mL EDC/NHS 1:1 containing 30 μL of PAMAM dendrimer 25% v/v, and incubated for 2 h in the ultrasound bath at a constant temperature (25±2° C.). Then, dispersions were centrifuged at 21,000 g for 10 min, washed three times with PBS for subsequent in vitro and in vivo experiments. Plasmids were incubated with dispersions of DGNP in a ratio 1:10 for 2 h in a rotatory shaker, centrifuged and washed three times with PBS before use for transfection and functional assays. Ratio of plasmid/nanoparticles was established using the variations in Zeta potential from positive (DGNP) to negative charge, when coating with plasmid, and evaluated with a Zetasizer nano ZS (Malvern Instruments Ltd., UK).

Physicochemical characterization of nanoparticles. Nanoparticle size was determined by dynamic light scattering (DLS), using a Zetasizer nano ZS (Malvern Instruments Ltd., UK). Measurements were carried out at 25° C. and at fixed angle of 173°, by analyzing the intensity of the scattered light supplied by a helium-neon laser (4 mW, λ=633 nm). DLS data were calculated from the autocorrelation function of scattered light by means of two mathematical approaches: the cumulants method and Dispersion Technology Software nano v. 5.10 (Malvern Instruments Ltd). Through the cumulants analysis, two important parameters were obtained: the mean hydrodynamic diameter (Z-Average) and the width of the particle size distribution (polydispersity index-PDI). To prepare samples for the measurements, 20 μL of graphite nanoparticle suspension were dispersed in 1480 μL of PBS, in an ordinary cuvette. Reported values of Z-Average and PDI corresponded to the average of approximately 40 measurement runs. The size and morphology of different nanoparticles were characterized by TEM, using a JEOL JEM 1010 microscope (JEOL, Akishima, Japan) equipped with an AMT XR40 digital imaging camera, at a magnification of 75000× and a maximum accelerating voltage of 100 kV. Particle diameter was determined in approximately 300 randomly selected nanoparticles from different TEM images using the morphometry software ImageJ v. 1.44 (U.S. National Institutes of Health, Bethesda, Md., USA). Osmolality was determined from osmometric depression of the freezing point (Advanced Instruments Osmometer 3300, Needham, HTs MA, USA).

Cell culture. Primary human umbilical vein endothelial cells (HUVECs) and mouse macrophages (RAW 264.7) were supplied by ATCC (Manassas, Va., USA). Human LX-2 hepatic stellate cells were a generous gift from Dr. Scott L Friedman. Dulbecco's phosphate buffered saline (DPBS), Dulbecco's Modified Eagle Medium (DMEM and penicillin/streptomycin were purchased from Thermo Fisher Scientific Inc. (Waltham, Mass., USA). HUVECs were cultured in pre-gelatinized plates with endothelial growth medium (EGM) supplemented with EGM-2 growth supplements (Lonza, Walkersville, Md.), 10% fetal bovine serum (FBS), and 50 U/mL penicillin/streptomycin. HUVECs were passaged when they reached 80% confluence and passages 2-5 were used for all experiments. RAW 264.7 mouse macrophages, isolated mouse hepatocytes and hepatic stellate cells were cultured with DMEM supplemented with 10% FBS. Human LX-2 cells were cultured with DMEM supplemented with 2% FBS. All cells were grown at 37° C. and 5% CO₂ in a water jacketed incubator.

Prolonged inflammation assay in macrophages. RAW 264.7 were seeded at 2×10⁴ cell/cm² density with complete DMEM medium supplemented with low FBS (1%) and incubated with TNF-α (5 ng/mL, Life Technologies, USA) for 7 days, with daily renovation of this pro-inflammatory medium. Cells were harvested at different time points; at day 0 (16 h after seeding with no TNF-α stimulation), and 1, 3, 5 and 7 days after TNF-α stimulation for RNA isolation or protein extraction using the TRIZOL kit (Gibco-Invitrogen, Paisley, UK) or lysis buffer containing 20 mM Tris-HCl, at pH 7.4, 1% Triton X-100, 0.1% SDS, 50 mM NaCl, 2.5 mM EDTA, 1 mM Na₄P₂O₇ 10H₂O, 20 mM NaF, 1 mM Na₃VO₄, 2 mM Pefabloc and a cocktail of protease inhibitors (Complete Mini, Roche) with protease inhibitors (Thermo Fisher, 87786) and phosphatase inhibitors (Thermo Fisher, 78420), respectively, for Real-time PCR and Western blot experiments.

Biological characterization of nanoparticles. Plasmid-DGNP cytotoxicity was analyzed on HUVECs using the CellTiter 96 Aqueous One Solution Cell Proliferation Assay (MTS assay, Promega, Madison, Wis., USA). Briefly, cells were seeded in pre-gelatinized 96-well plates at a cell density of 5×10³ cells per well, serum starved for 6 h and then incubated with plasmid-DGNP at different concentrations (500, 50 and 5 μg/mL) for 24 h. Just before determination of cell viability, cells were washed with PBS and transferred into starvation medium. Cytotoxicity was determined by adding 20 μL of MTS solution to each well. After 2 h, the absorbance was measured at 490 nm using a microplate spectrophotometer (Varioskan Flash spectrophotometer, Thermofisher Scientific). Cell viability was expressed as the absorbance of cells treated with plasmid-DGNP relative to cells treated with PBS (control). Each condition was performed in quadruplicates and reported as mean±SD.

Uptake kinetics of plasmid-DGNP. RAW 264.7 mouse macrophages were cultured with DMEM with 10% FBS in 24 wells (10⁵ cells/well) for 24 h and then serum-starved for 6 h. Afterwards cells were incubated with plasmid-DGNP 100 ng/mL in the presence or absence of TNF-α (5 ng/mL) and images were taken at different time points (30, 60, 120 and 180 min) with a light microscope. Black aggregates of plasmid-DGNP were visualized at high magnification to establish the number of cells incorporating plasmid-DGNP. Percentage of cells incorporating plasmid-DGNP is calculated with the formula: number of cells with black aggregates/total number of cells per field×100. At least 30 different fields were used to calculate the uptake percentage per time point.

Intracellular localization of FITC-DGNP in macrophages. DGNP (10 μg/mL) were incubated with FITC (2 mg/mL, Sigma) for 1 h at room temperature in the dark. Afterwards FITC-DGNP were centrifuged at 21000 Gs for 10 min, washed three times with DMSO, and then three times with PBS for subsequent in vitro experiments. FITC-DGNP were incubated with inflamed RAW 264.7 mouse macrophages for 24 h, washed with PBS, and visualized with an epifluorescence microscope (Fluo Zeiss Axio Observer Z1, Zeiss, Oberkochen, Germany) and a digital imaging system (Ret Exi, Explora Nova, La Rochelle, France). DAPI was used as mounting medium to counterstain cell nuclei.

Functional assay of plasmid transfection efficiency and M2-like subset switch. The transfection efficiency of plasmid-DGNP complexes was studied in inflamed RAW 264.7 macrophages. Cells were seeded at a concentration of 5×10⁴ cells in 2-well Labtek II chamber slides, grown to 80% confluence, and inflamed with TNF-α (5 ng/mL) for 16 h. After that, cells were serum-starved for 6 h and incubated for 3 h with 100 ng/mL plasmid-DGNP containing 10 ng/mL of plasmid DNA expressing RNF41 or EGFP reporter. Cells were then washed and incubated for 3 days. Afterwards, cells were washed with PBS and mounted with a coverslip using a DAPI mounting medium to counterstain cell nuclei. Intracellular presence of synthesized EGFP was visualized with an epifluorescence microscope. To analyze the possible switch from M1 to M2 macrophages, cells were stained with rabbit polyclonal anti-mannose receptor (1:100, Abcam, Cambridge, Mass.) and revealed with Cy3-conjugated donkey-anti-rabbit IgG (Jackson ImmunoResearch Laboratories, West Grove, Pa., USA) incubated for 1 h at room temperature. The presence of synthesized mannose receptor was visualized with an epifluorescence microscope.

Collagen degradation assay. Gelatin and FITC were obtained from Sigma (St. Louis, Mo.). The preparation of FITC-conjugated gelatin and the quantitative analysis of collagen degradation assay was performed as described in Melgar-Lesmes, P. et al. Graphene-Dendrimer Nanostars for Targeted Macrophage Overexpression of Metalloproteinase 9 and Hepatic Fibrosis Precision Therapy. Nano Lett 18, 5839-5845 (the teachings of which are incorporated by reference in their entirety). Briefly, gelatin was dissolved (1 mg/mL) in a buffer containing 61 mM NaCl and 50 mM Na2B4O7 (pH 9.3) and then incubated at 37° C. for 1 h. After this incubation period, FITC was added (2 mg/mL) and mixed for 2 h in complete darkness. This mixture was then dialyzed at RT in PBS in complete darkness for 4 days with 2-3 PBS changes per day. After a quick spin to remove insoluble material, small aliquots were stored in the dark at 4° C. FITC-conjugated gelatin-coated plates were prepared covering the surface of each well with FITC-gelatin and fixed with 1 drop of 0.5% ice-cold formaldehyde in PBS at 4° C. for 15 min. Wells were then gently washed three times with PBS and finally quenched in complete medium for 1 h at 37° C. Cells were cultured for variable lengths of time up to 7 days and supernatants were collected. Cells were fixed, washed, stained with mounting medium containing DAPI and visualized with an epifluorescence microscope. Supernatants were centrifuged and fluorescence quantified with a Hitachi F-2500 Fluorescence Spectrophotometer (Hitachi High Technologies Corp., Tokyo, Japan).

Animal Studies. Male Balb/c mice were purchased from Charles River Laboratories (Charles River, Saint Aubin les Elseuf, France). All animals were maintained in a temperature-controlled room (22° C.) on a 12-h light-dark cycle. The study was performed according to the criteria of the Investigation and Ethics Committees of the Hospital Clinic and University of Barcelona. After arrival, mice were continuously fed ad libitum until euthanasia. To induce liver cirrhosis, mice were injected intraperitoneally twice a week with CCl₄ diluted 1:8 v/v in corn oil for 9 weeks. Dispersions of plasmid-DGNP were then intravenously injected (50 μg/Kg in a ratio plasmid/DGNP 1:10) every 3 days. Animals were euthanized after 10 days of treatment. Liver samples and serum were collected and frozen for further analysis. Serum parameters were measured using a BS-200E Chemistry Analyzer (Mindray Medical International Ltd, Shenzhen, China).

Partial hepatectomy (70% in healthy and 40% in cirrhotic mice) was performed as described in Mitchell, C. & Willenbring, H. A reproducible and well-tolerated method for ⅔ partial hepatectomy in mice. Nat Protoc 3, 1167-1170 (the teachings of which are incorporated by reference in their entirety). Hepatectomy was performed at 40% in cirrhotic mice to avoid unnecessary animal losses of these already diseased animals according to the criteria of the Investigation and Ethics Committees of the Hospital Clínic and University of Barcelona. Dispersions of plasmid-DGNP were intravenously injected to hepatectomized animals, which were euthanized 7 days after hepatectomy to obtain and analyze tissue and serum samples as described above.

Isolation of hepatic CD11b-macrophages, stellate cells, and hepatocytes. Freshly isolated primary hepatic CD11b-macrophages were obtained from the livers of control and cirrhotic patients and mice. Briefly, hepatocytes, macrophages and hepatic stellate cells were purified after collagenase A (Roche Diagnostics, Basel, Switzerland) administration via retrograde perfusion in mice or via an intravenous catheter in human liver samples, and subsequent Histodenz gradient (Sigma-Aldrich) as described in Mederacke, I., Dapito, D. H., Affo, S., Uchinami, H. & Schwabe, R. F. High-yield and high-purity isolation of hepatic stellate cells from normal and fibrotic mouse livers. Nat Protoc 10, 305-315 (the teachings of which are incorporated by reference in their entirety). Purification was optimized using CD11b magnetic beads (MACS system, Miltenyi Biotec, Bergisch-Gladbach, Germany) using a modified protocol reported in Ribera, J. et al. A small population of liver endothelial cells undergoes endothelial-to-mesenchymal transition in response to chronic liver injury. Am J Physiol Gastrointest Liver Physiol 313, G492-G504 (the teachings of which are incorporated by reference in their entirety). The fractions corresponding to CD11b+-macrophages were resuspended in TRIZOL (Gibco-Invitrogen, Paisley, UK) for total RNA extraction. A 0.5 μg aliquot of total RNA was reverse transcribed using a complementary DNA synthesis kit (High-Capacity cDNA Reverse Transcription Kit, Applied Biosystems, Foster City, Calif., USA) for further analysis of gene expression using Real-Time PCR.

Proliferation assay in isolated hepatocytes incubated with macrophage-derived conditioned medium. Effects of macrophage-derived conditioned medium from RAW 264.7 macrophages treated with the different plasmid-DGNP were analyzed in mouse isolated hepatocytes using the CellTiter 96 Aqueous One Solution Cell Proliferation Assay (MTS assay, Promega, Madison, Wis., USA). Isolated mouse hepatocytes were seeded in 96-well plates at a cell density of 5×10³ cells per well, serum starved for 6 h, washed with PBS and then incubated with fresh starving medium mixed with conditioned medium 1:1 from RAW 264.7 macrophages treated with PBS, 10% FBS, TNF-α+pScramble-DGNP, TNF-α+pRNF41-DGNP, TNF-α+pshRNF41-DGNP for 3 days. Conditioned medium was centrifuged, and supernatants stored at −80° C. for proliferation assays. Conditioned medium from macrophages treated with pRNF41-DGNP was mixed with IGF-1 antibody (2 μg/mL) for 2 h before the proliferation assay to evaluate its involvement in the proliferation of hepatocytes. Final conditioned medium mixtures were incubated with hepatocytes for 24 h. Proliferation was determined by adding 20 μL of MTS solution to each well. After 2 h, the absorbance was measured at 490 nm using a microplate spectrophotometer (Varioskan Flash spectrophotometer, Thermofisher Scientific). Cell viability was expressed as absorbance and compared to the absorbance of cells receiving an equal volume of PBS (control). Each condition was performed in sextuplicate and reported as mean±SD.

Western Blot. Total protein was extracted from cells with lysis buffer containing 20 mM Tris-HCl, at pH 7.4, 1% Triton X-100, 0.1% SDS, 50 mM NaCl, 2.5 mM EDTA, 1 mM Na₄P₂O₇ 10H₂O, 20 mM NaF, 1 mM Na₃VO₄, 2 mM Pefabloc and a cocktail of protease inhibitors (Complete Mini, Roche) and phosphatase inhibitors (Thermo Fisher, 78420). Proteins were separated on a 10% SDS-polyacrylamide gel (Mini Protean III, BioRad, Richmond, Ca) and transferred for 2 h at 4° C. to nitrocellulose membranes of 0.45 μm (Transblot Transfer Medium, BioRad, Richmond, Calif.) that were stained with Ponceau-S red as a primary control for protein loading. The membranes were incubated at 4° C. overnight with the following antibodies: rabbit anti-pAkt (Ser127) and anti-Akt (1:1000, Cell Signaling), rabbit anti-phospho-Erk and anti-Erk (1:1000, Cell Signaling) and β-actin as loading control. Next, the membranes were incubated with a donkey ECL-anti-rabbit IgG peroxidase-conjugated secondary antibody at 1:2000 dilution (GE Healthcare) for 1 h at room temperature. The bands were visualized using Chemidoc Imaging System (Biorad Laboratories, Inc).

Immunofluorescence and imaging in tissues. Liver was excised and tissue was washed with PBS and fixed with 10% buffered formaldehyde solution for 24 h. Afterwards the tissue was cryo-protected with 30% sucrose solution (in PBS) and then embedded using Tissue-Tek OCT compound (Sakura Fineteck USA, Torrance, Calif.) and frozen. Liver sections underwent 1% SDS solution antigen retrieval for 5 min at room temperature and then were blocked with 5% normal goat serum. Liver sections were incubated with rabbit polyclonal anti-mannose receptor (1:100, Abcam, Cambridge, Mass.), rabbit polyclonal anti-PCNA antibody (1:50, Abcam, Cambridge, Mass.) and rat anti-Ly-6C monoclonal IgG antibody (1:100, Thermofisher Scientific). Primary antibodies were revealed with Cy3-conjugated donkey-anti-rabbit IgG (1:500, Jackson ImmunoResearch Laboratories, West Grove, Pa., USA); Cy3-conjugated donkey anti-rat IgG (1:500, Jackson ImmunoResearch Laboratories, West Grove, Pa., USA) or donkey-anti-rabbit IgG Alexa Fluor 594 (1:500, Jackson ImmunoResearch Laboratories, West Grove, Pa., USA) incubated for 1 h at room temperature. The presence of mannose receptor, Ly6C or PCNA was visualized with an epifluorescence microscope. DAPI (Vectashield, Vector laboratories, Burlingame, Calif.) was used to counterstain cell nuclei.

Fibrosis quantification. Liver was excised and tissue was washed with PBS and fixed with 10% buffered formaldehyde solution for 24 h. Afterwards, the tissue was embedded using paraffin. Before staining, paraffin was removed using xylene, ethanol, and deionized water. Liver sections (4 μm) were stained in 0.1% Sirius Red F3B (Sigma) with saturated picric acid (Sigma). Relative fibrosis area (expressed as a percentage of total liver area) was analyzed in 20 fields of Sirius Red-stained liver sections per animal using the morphometry software ImageJ v 1.44. To evaluate the relative fibrosis area, the measured collagen area was divided by the net field area and then multiplied by 100. From each animal analyzed, the amount of fibrosis as percentage was measured and the average value presented.

Gene expression assay with Real-Time PCR. Total RNA from liver was extracted using commercially available kits: RNeasy (Gibco-Invitrogen, Paisley, UK). A 0.5 μg aliquot of total RNA was reverse transcribed using a complementary DNA synthesis kit (High-Capacity cDNA Reverse Transcription Kit, Applied Biosystems, Foster City, Calif., USA). Primers and probes for gene expression assays (Applied Biosystems) were selected as follows: RNF41 (Taqman assay reference from Applied Biosystems: Human: Hs01086974_m1; Mouse: Mm01159897_m1), USP8 (Human: Hs00987105_g1; Mouse: Mm00451077_m1), IGF-1 (Mm00439560_m1), HGF (Mm01135184_m1), TIMP1 (Human: Hs01092512_g1; Mouse: Mm01341360_g1), ACTA2 (α-SMA, Human: Hs00426835_g1; Mouse: Mm01204962_gH), COL1A1 (Human: Hs00164004_m1; Mouse: Mm00801666_g1), OSM (Mm01193966_m1), PDGF-BB (Mm00440677_m1), TGF-β (Mm01178820_m1), MMP-9 (Mm00442991_m1), IL-10 (Mm00439614_m1), CD36 (Mm00432403_m1), NOS2 (Mm00440502_m1), COX-2 (Mm00478374_m1), IL1-β (Mm00434228_m1), ARG-1 (Mm00475988_m1), MRC1 (Mm00485148_m1), RETN1A (Mm00445109_m1), and mouse hypoxanthine phosphoribosyl transferase (HPRT, Mm03024075_m1) and human glyceraldehyde-3-phosphate dehydrogenase (GAPDH, Hs02786624_g1), used as endogenous standards. Expression assays were designed using the Taqman Gene Expression assay software (Applied Biosystems). Real-time quantitative PCR was analyzed in duplicate and performed with a Lightcycler-480 II (Roche Diagnostics). A 10 μl aliquot of the total volume reaction of diluted 1:8 cDNA, Taqman probe and primers and FastStart TaqMan Master (Applied Biosystems) were used in each PCR. The fluorescence signal was captured during each of the 45 cycles (denaturing 10 s at 95° C., annealing 15 s at 60° C. and extension 20 s at 72° C.). Water was used as a negative control. Relative quantification was calculated using the comparative threshold cycle (CT), which is inversely related to the abundance of mRNA transcripts in the initial sample. The mean CT of duplicate measurements was used to calculate ΔCT as the difference in CT for target and reference. The relative quantity of the product was expressed as fold induction of the target gene compared with the control primers according to the formula 2-ΔΔCT, where ΔΔCT represents ΔCT values normalized with the mean ΔCT of control samples.

Statistical analysis. All data were expressed as mean±standard deviation (SD). Statistical analysis of the results was performed by one-way analysis of variance (ANOVA) with the posthoc Newman-Keuls test or by Student's t-tests, where appropriate (GraphPad Prism v6.0a). Differences were considered statistically significant at a p-value≤0.05.

### Example 2. Macrophage RNF41 Expression is Downregulated in Liver Cirrhosis

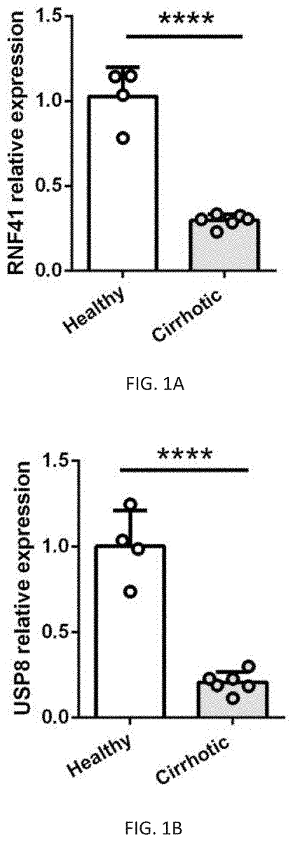

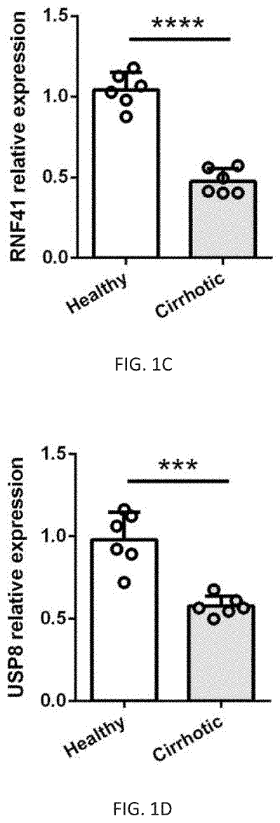

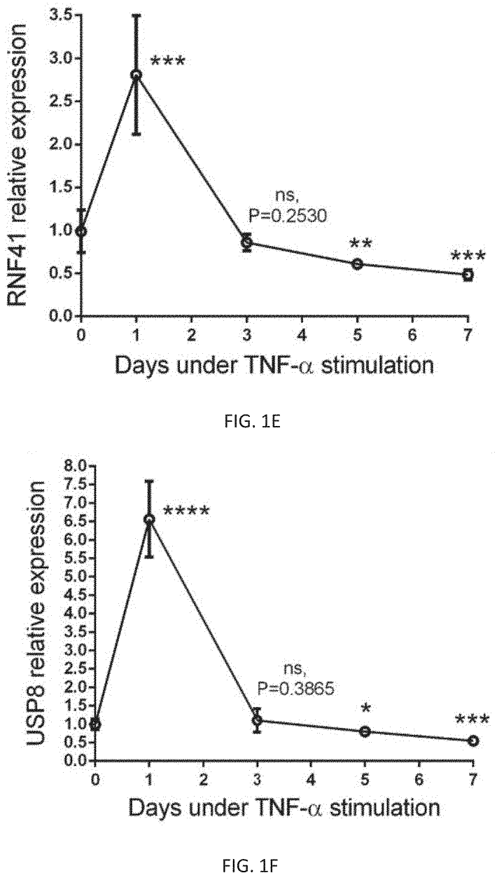

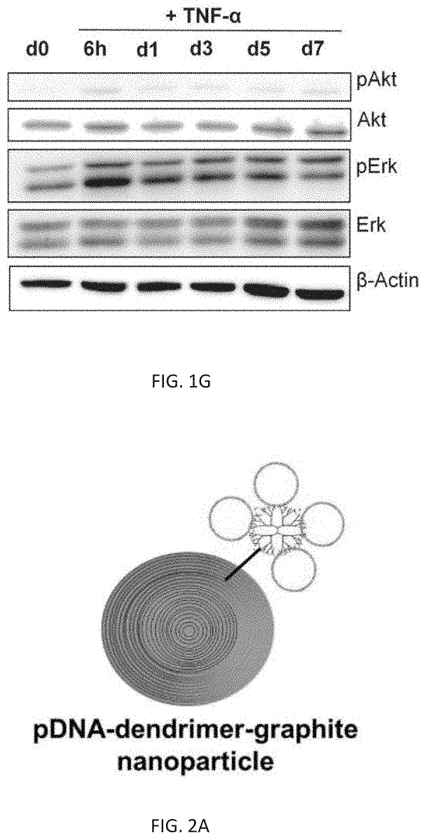

To determine the expression levels of RNF41 in those macrophages recruited to the cirrhotic liver, CD11b+ macrophages from liver biopsy specimens of patients with liver cirrhosis and healthy subjects was isolated. This cell surface marker was selected for isolation because it is a selective marker of macrophage attachment and function during liver injury and regeneration. Six participants were selected (one female and five males, 58.5±5.1 years) with decompensated liver cirrhosis and MELD scores between 19 and 30 from a single center (Hospital Clinic of Barcelona, Spain). The demographic and baseline characteristics of study participants are shown in Table 1 (below). Participants in the diseased group displayed a mean duration of cirrhosis of 5.8±8 years. The cirrhotic milieu influenced macrophage expression-RNF41 single-cell mRNA expression was significantly lower in macrophages from cirrhotic than healthy subjects (FIG. 1A) regardless of cirrhosis etiology (alcoholic, non-alcoholic or autoimmune). This also occurred with ubiquitin specific peptidase 8 (USP8) expression, a known stabilizer of RNF41 activity (FIG. 1B).

TABLE 1
Data is shown as Mean ± Standard Deviation, or Number
of Participants (Percentage, %). BMI: Body Mass Index;
NASH: Non-Alcoholic Steatohepatitis; PBC: Primary Biliary
Cirrhosis; MELD: Model for End-Stage Liver Disease

| | Cirrhotic | Healthy |
|---|---|---|
| Variables | N = 6 | N = 4 |
| Age (years) | 58.5 ± 5.1 | 56.75 ± 26.4 |
| Gender | | |
| Male | 5 (83.3) | 1 (25) |
| Female | 1 (16.7) | 3 (75) |
| BMI (kg/m²) | 27.8 ± 4 | 25.7 ± 1.7 |
| Etiology of liver disease | | |
| Alcoholic | 3 (50) | |
| Alcoholic + NASH | 2 (33.3) | |
| PBC | 1 (16.7) | |
| Child-Pugh Score | | |
| A | 1 (16.7) | |
| B | 0 (0) | |
| C | 5 (83.3) | |
| MELD score | 23 ± 4.7 | |
| Cirrhosis duration (years) | 5.8 ± 8 | |

Due to hepatic chronic inflammation occurring in liver cirrhosis resulting in downregulation of macrophage RNF41, cross-talk between RNF41 and cytokine receptors was evaluated. To evaluate the role of macrophage RNF41 on an animal model of liver cirrhosis the BALB/c mouse strain was selected as it is most sensitive to induction of liver fibrosis. Hepatic chronic inflammation and cirrhosis was induced by intraperitoneal injection administration of CCl4 0.5 μL/g of body weight for 9 weeks which classically promotes the formation of hepatic regenerative nodules surrounded by fibrotic tracts composed by dense collagen bundles and pro-inflammatory infiltrated macrophages. Quantification of RNF41 in CD11b+ macrophages from liver specimens obtained from healthy and cirrhotic mice mirrored results in human specimens with down-regulated expression of RNF41 (FIG. 1C) and USP8 (FIG. 1D).

The mouse BALB/c-derived macrophage cell line RAW 264.7 was used to find out how mouse macrophages respond to prolonged inflammation induced by TNF-α, a prominent cytokine driving inflammation in chronic liver disease, in terms of RNF41 regulation. RNF41 expression was up-regulated in mouse RAW 264.7 macrophages during the first 24 h of induction with TNF-α, then decreased up to day 5 and resulted significantly lower compared to untreated macrophages afterwards (FIG. 1E). The same pattern of initial up-regulation and subsequent dropped expression after day 5 was observed with USP8, the RNF41 stabilizer (FIG. 1F). It is known that phospho-Akt (pAkt) phosphorylates USP8 and this latter stabilizes RNF41. To understand the connection between the inflammatory activity of TNF-α and RNF41, the downstream transduction pathways engaged by TNF-α including Akt and mitogen-activated protein (MAP) kinases (such as extracellular-signal-regulated kinases (ERKs) was analyzed. Phosphorylation of Erk1/2 and Akt increased during the first 6 h after TNF-α stimulation, but only pAkt substantially dropped afterwards (FIG. 1G) coinciding with the observed down-regulation pattern of RNF41 and its stabilizer USP8.

### Example 3. Graphite Nanoparticles Linked to PAMAM Dendrimer are a Biosafe Selective Gene Therapy Nanosystem for RNF41 Plasmid-Induced Expression in Inflammatory Macrophages

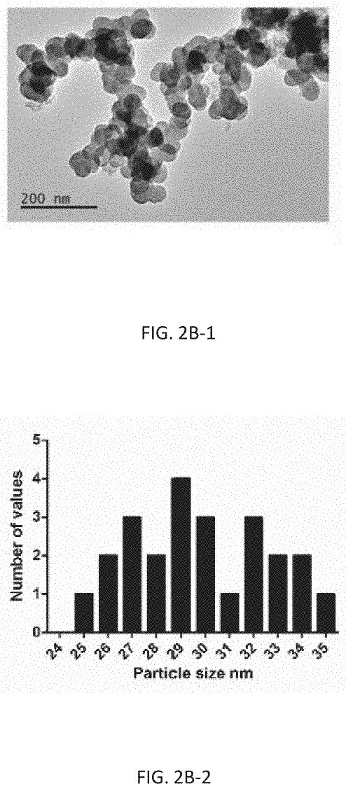

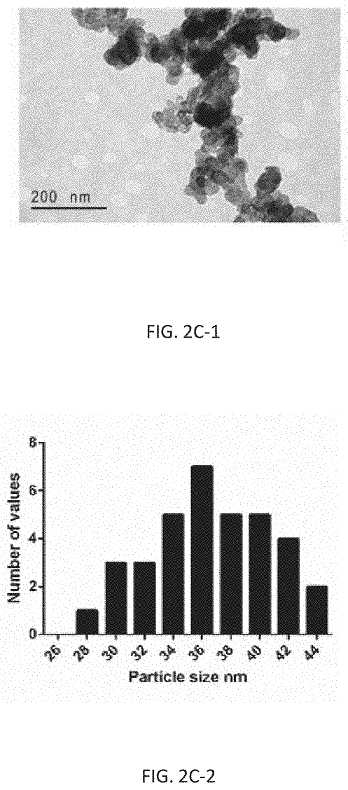

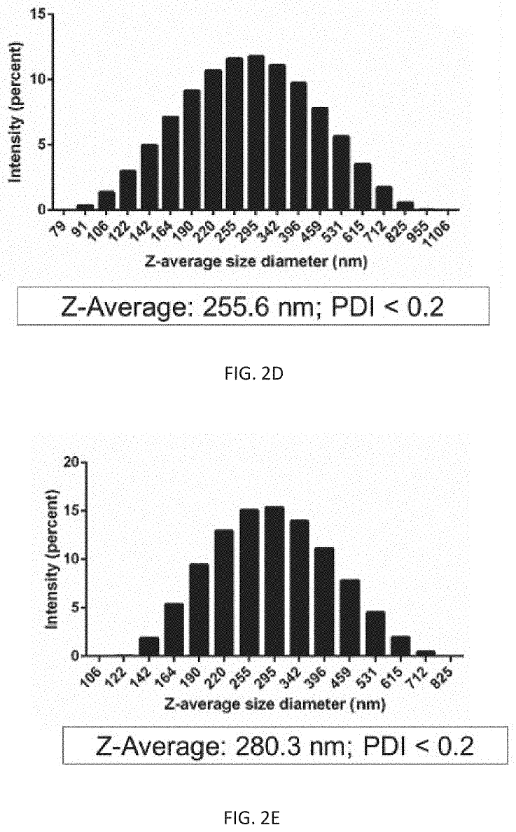

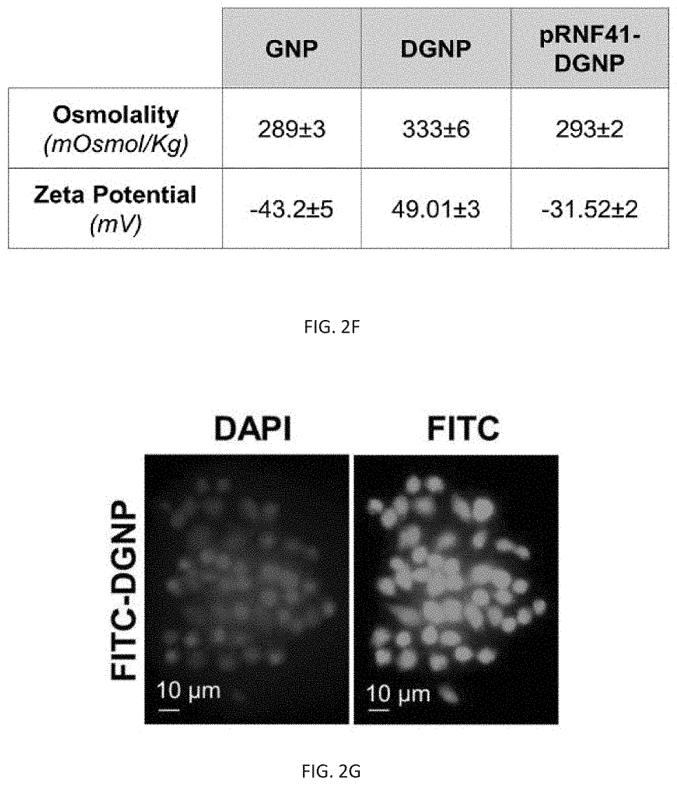

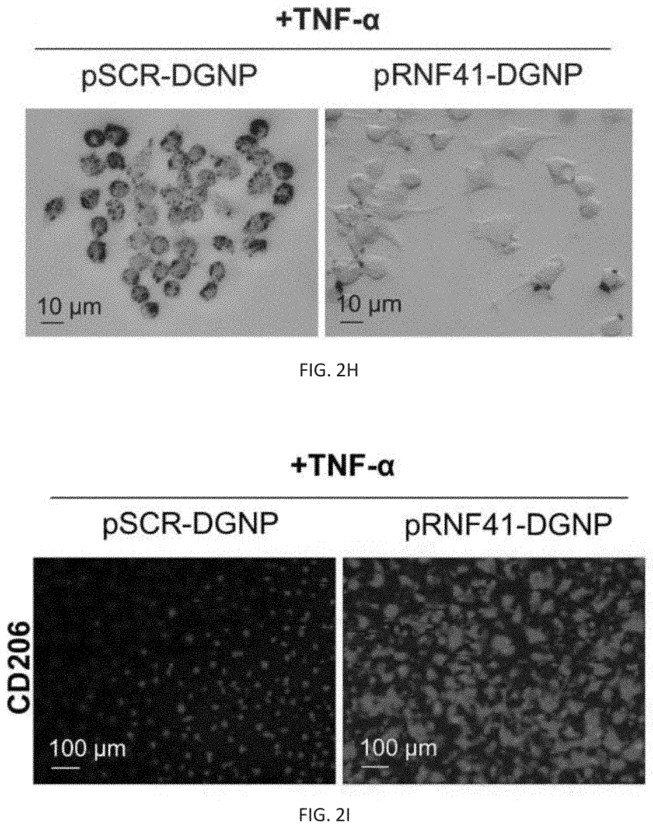

An expression plasmid for RNF41 was designed with a CD11b promoter (to assure that only recruited inflammatory macrophages can express this protein) and a gene reporter (EGFP) under the control of an immediate early enhancer and CMV promoter (FIG. 7A). To synthesize the gene therapy nanosystem, graphite nanoparticles (GNP) were first oxidized to obtain GNP decorated with carboxylated surface. Then, PAMAM generation 5 dendrimers, which are established for the binding of nucleic acids (such as plasmids) by electrostatic forces as well as for their transport and release, were chemically attached (FIG. 2A). Transmission Electron Microscopy (TEM) images revealed GNP with a diameter of 29.9±2.9 nm (FIG. 2B-1 and FIG. 2B-2) that rose to 36.8±4.2 nm when PAMAM dendrimers were covalently incorporated (FIG. 2C-1 and FIG. 2C-2). GNP diameter visualized by TEM was more than eight times smaller than the size of nanoparticles dispersed in PBS and measured by Dynamic Light Scattering (DLS). Indeed, hydrodynamic diameter of GNP resulted in a mean particle diameter (Z-average) of 255.6 nm, denoting a highly hydrated corona and a high aggregation of GNP in PBS with rather narrow particle size distributions (polydispersity index, PDI<0.20) (FIG. 2D). Z-average of dendrimer-GNP (DGNP) increased to 280.3 nm preserving a narrow particle size distribution (FIG. 2E). GNP showed a negative zeta potential (−43.2 mV) due to the carboxylic groups and biologically interesting isotonic properties (FIG. 2F). The chemical binding of dendrimers to GNP promoted a zeta potential switch to positive (49.01 mV) resulting in hypertonic nanoparticle dispersions (FIG. 2F). The addition of a RNF41 plasmid to DGNP (pRNF41-DGNP) switched zeta potential back to negative (−31.52 mV), returning the composition to physiological osmolality (FIG. 2F). The phosphate backbone of pDNA is negatively charged due to the bonds created between the phosphorous atoms and the oxygen atoms. Each phosphate group contains one negatively charged oxygen atom, therefore the entire strand of DNA is negatively charged due to repeated phosphate groups. This negative charge is linking to positively charged dendrimers to finally switch zeta potential charge to negative.

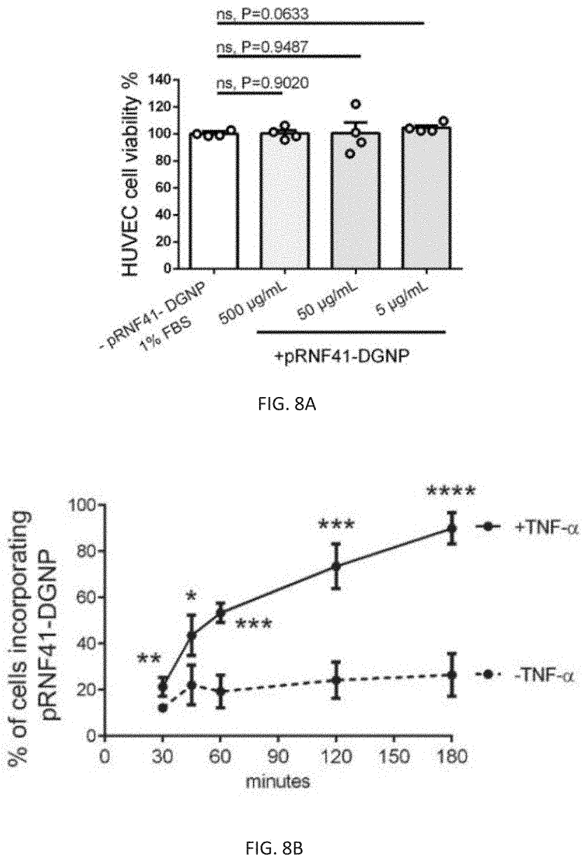

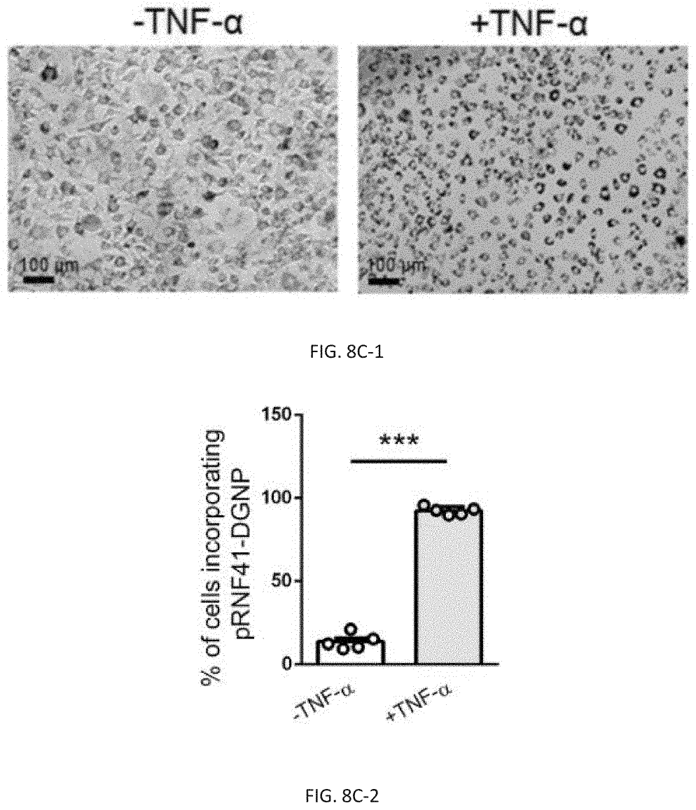

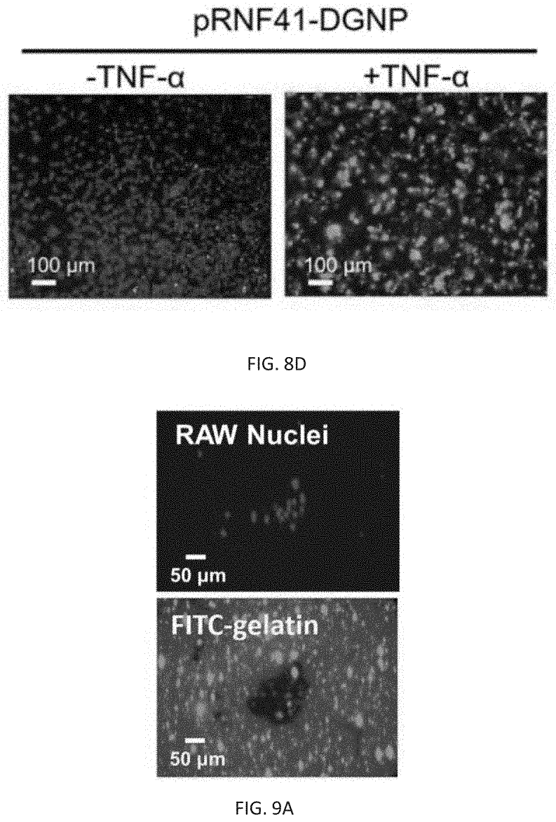

Isotonic dispersions of pRNF41-DGNP were then tested for biocompatibility on human endothelial cells, as the standard primary cell barrier in blood vessels, and consequently, the first biological point of contact with an intravenously administered formulation. There were no harmful effects on human umbilical vein endothelial cells (HUVEC) exposed over 24 h to nanoparticle concentrations ranging from 5 to 500 μg/mL (FIG. 8A). The uptake of most nanoparticles over 200 nm administered in vivo is widely-accepted to involve mainly macrophages and especially pro-inflammatory macrophages at diseased sites. The incorporation of pRNF41-DGNP in RAW 264.7 macrophages activated by TNF-α increased over time up to 45 min and then only rose in the presence of TNF-α, reaching most of cells after 180 min (FIG. 8B) and lasted for at least 24 h (FIG. 8C-1 and FIG. 8C-2), indicating that the GNP core is involved in the selective macrophage uptake. FITC-decorated DGNP confirmed the intracellular fate of dendrimers after internalization in inflamed macrophages. After 24 h of incubation, FITC-DGNP were internalized and degraded by macrophages, distributing the dendrimer-FITC molecules throughout the cell, including the cell nucleus (FIG. 2G). These results suggest that these nanoparticles could be useful for the selective pDNA-dendrimer incorporation by pro-inflammatory macrophages present in chronically inflamed livers to deliver an effective gene therapy. Certainly, dendrimers are known to escape from lysosomes by the proton sponge effect, opening pores in the nuclear membrane for pDNA or siRNA gene therapy.

The effectivity of plasmid-DGNP as gene therapy was confirmed in vitro where pRNF41-DGNP were mainly phagocytized by macrophages stimulated with TNF-α, and plasmid expression efficiency was functionally highlighted by the presence of high levels of intracellular EGFP in most of cells after 3 days of incubation (FIG. 8D). Moreover, macrophages incubated with pRNF41-DGNP and TNF-α (a CD11b promoter activator) displayed a clear switch in macrophage morphology (FIG. 2H) and phenotype, exemplified by elevated expression of CD206 (mannose receptor, a M2-like macrophage marker) (FIG. 2I).

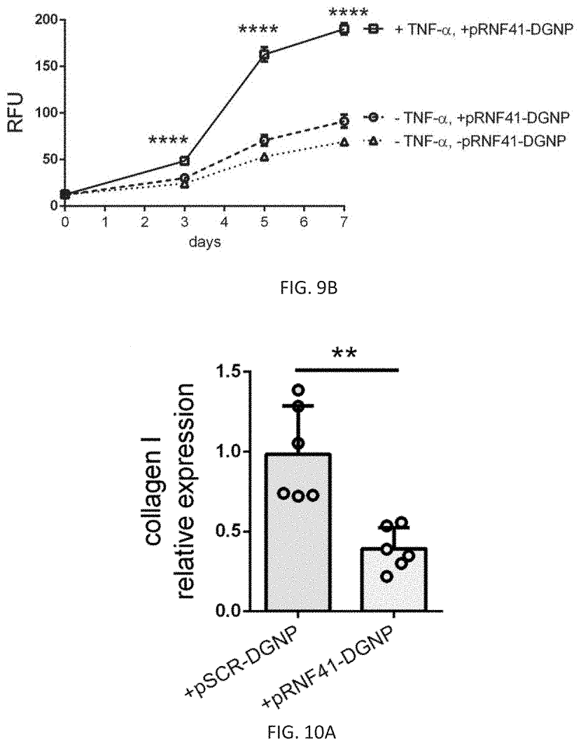

M2-like macrophages produce high amounts of matrix metalloproteinases to degrade extracellular matrix proteins such as collagen. As this collagenase activity of M2-like macrophages might be beneficial for the treatment of liver fibrosis, the capacity of macrophages treated with pRNF41-DGNP to digest collagen using FITC-gelatin was tested, not only as a function of RNF41 overexpression, but also as a sign of phenotypical transformation to M2 macrophages. RAW 264.7 macrophages seeded on FITC-gelatin coated plates and treated with pRNF41-DGNP for 5 days displayed a black halo because of the collagen digestion and a green nuclear staining due to the EGFP expression (FIG. 9A). A time-course quantitative analysis of FITC released to the medium during the gelatinase assay revealed that collagen degradation exponentially increased due to pRNF41-DGNP 3 days after incubation only when TNF-α was present (FIG. 9B). Collagen degradation was much lower with or without pRNF41-DGNP in the absence of inflammatory stimulus (FIG. 9B). pRNF41 degrades more than non-treated and non-inflamed macrophages, likely due to basal levels of expression of CD11b, which is the promoter of the plasmid. It is significant (p<0.01). This functional experiment was the rationale for Example 4's administration schedule of 3 days for in vivo experiments in animals with liver fibrosis using pRNF41-DGNP.

### Example 4. RNF41 Restoration in Inflammatory Macrophages Orchestrates Fibrosis Regression and Hepatocyte Proliferation

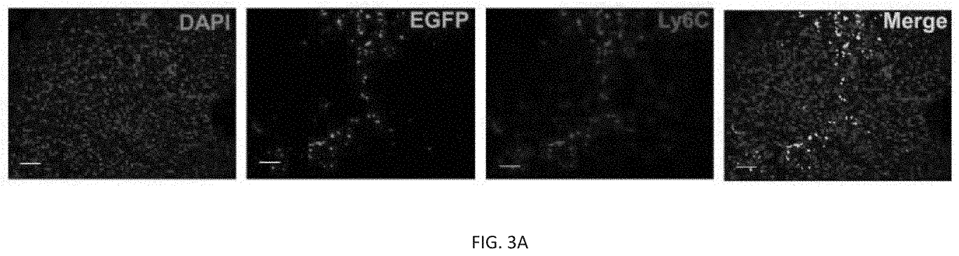

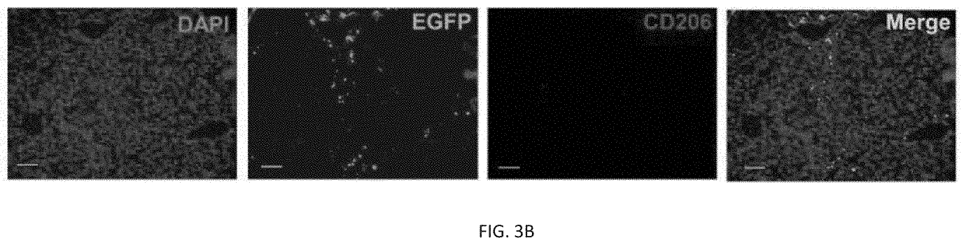

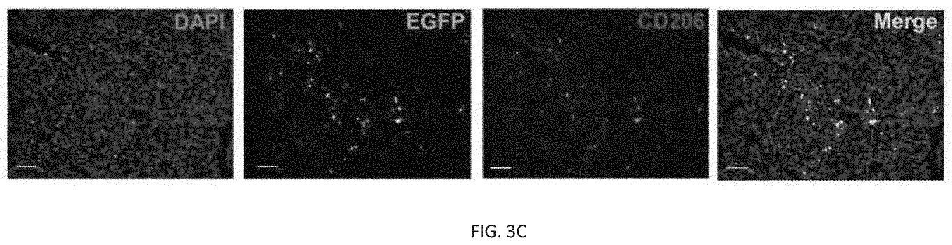

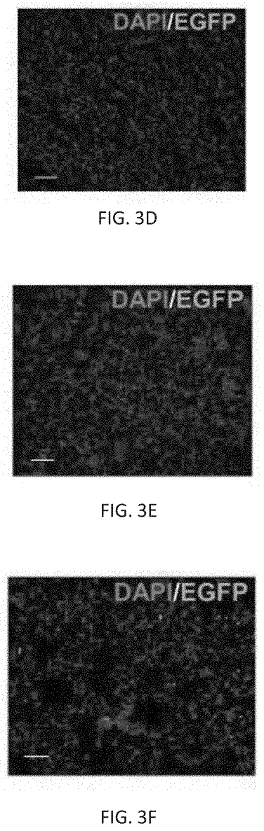

pRNF41-DGNP or DGNP with an equal plasmid but with a RNF41-scrambled sequence (pSCR-DGNP) was administered intravenously every 3 days for 10 days to mice with CCl₄-induced liver cirrhosis. It was determined that pDNA-DGNP was incorporated into hepatic inflammatory macrophages in cirrhotic mice such as in our in vitro studies. Immunofluorescence staining of hepatic EGFP-positive cells in cirrhotic mice treated with pSCR-DGNP showed high intracellular EGFP levels specifically in Ly6c-stained inflammatory macrophages (a M1 macrophage surface marker) (FIG. 3A). Indeed, these EGFP-positive M1-like macrophages were lacking for CD206 all along the fibrotic tracts in cirrhotic livers (FIG. 3B) compared to macrophages expressing both EGFP and CD206 in liver fibrotic tracts from animals treated with pRNF41-DGNP (FIG. 3C). The presence of functional pSCR-DGNP was negligible in other organs such as kidney (FIG. 3D), and very low in spleen (FIG. 3D) and lung (FIG. 3F) from cirrhotic animals, denoting the high selectivity of these nanoparticles for inflammatory macrophages present in injured livers.

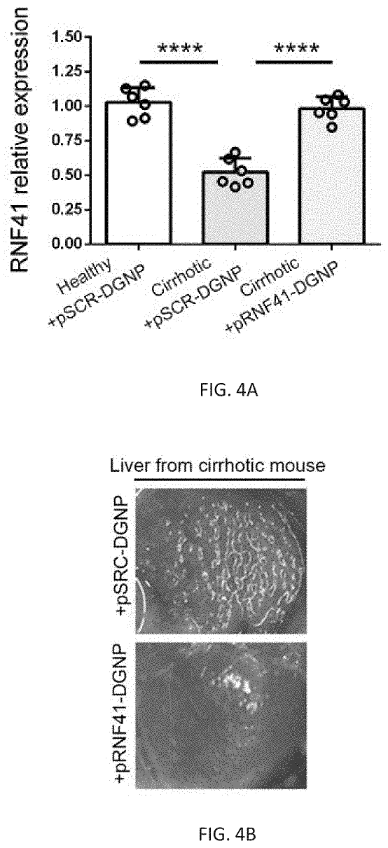

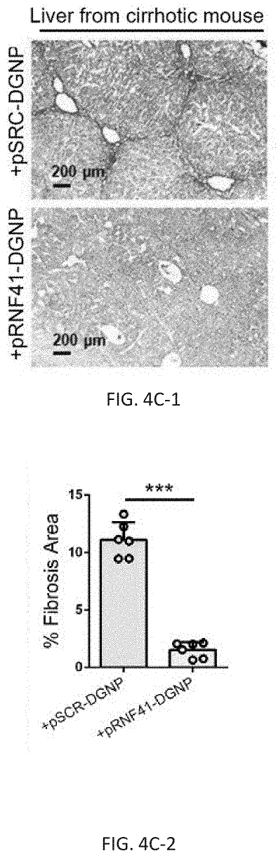

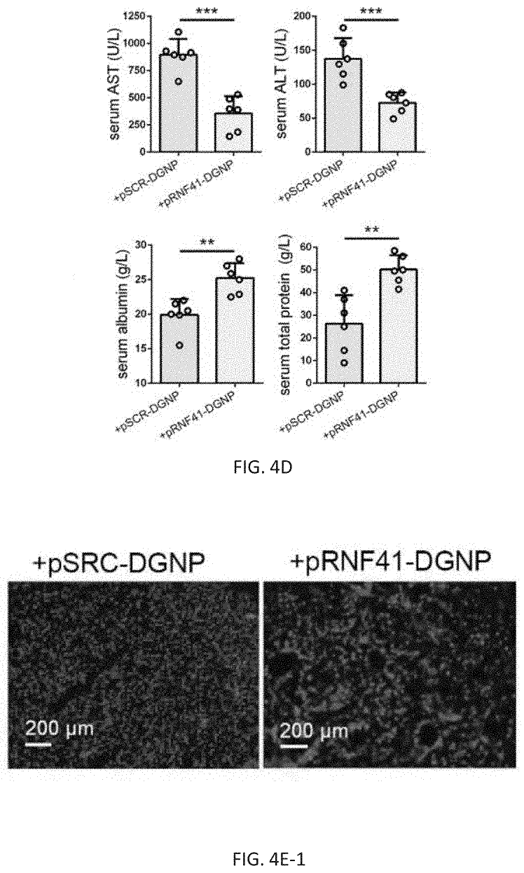

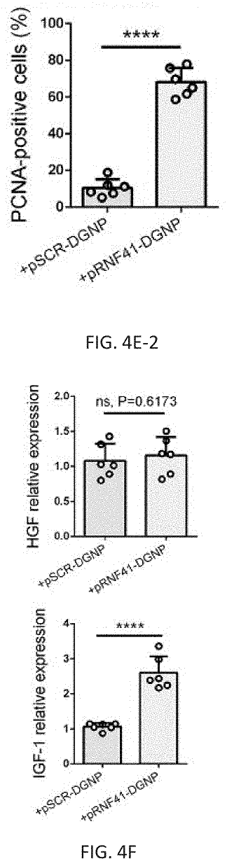

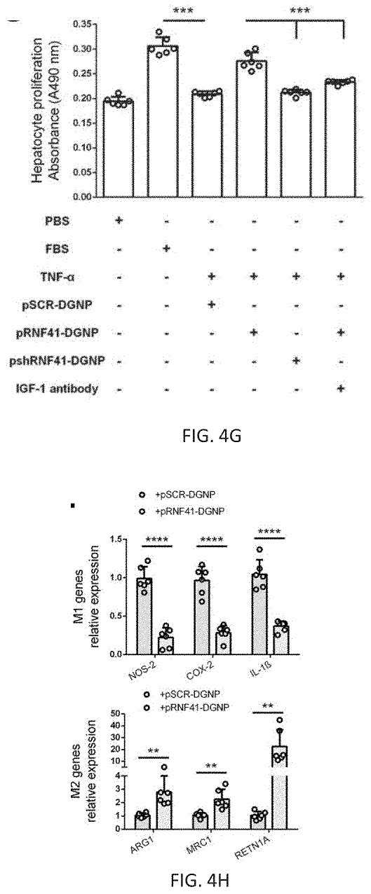

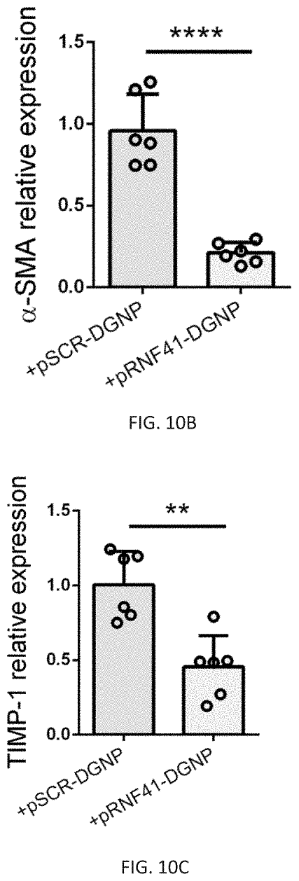

Hepatic macrophages from healthy mice treated with pSCR-DGNP, cirrhotic mice treated with pSCR-DGNP, and cirrhotic mice treated with pRNF41-DGNP were isolated and analyzed for relative RNF41 expression. As shown in Example 2, in untreated cirrhotic mice or in patients with liver cirrhosis, macrophage expression of RNF41 was significantly reduced in animals with cirrhosis treated with pSCR-DGNP compared to controls and restored to physiological levels after pRNF41-DGNP exposure (FIG. 4A). The first hepatic effect of macrophage RNF41 recovery after plasmid administration was visually appreciated as a change in the macroscopic aspect of cirrhotic liver from micronodular pathology to a non-fibrotic liver appearance (FIG. 4B). Regenerative nodules characteristically occur in the cirrhotic liver and therefore are referred to as cirrhotic nodules. In this setting, regenerative nodules are surrounded by fibrous septa. Roundish and sharply circumscribed, they are numerous and diffusely distributed throughout the liver. Indeed, rescue of RNF41 expression in macrophages of cirrhotic liver promoted an 86% reduction in the hepatic fibrosis area and a recovery of physiological parenchymal structure (FIG. 4C-1 and FIG. 4C-2). This decrease in the collagen fibers in the cirrhotic liver was associated with fall in collagen-I expression (FIG. 10A) and mitigated activity of hepatic stellate cells (HSC), which was illustrated by a reduced expression of alpha smooth muscle actin (α-SMA) (FIG. 10B) and tissue inhibitor of metalloproteinases-1 (TIMP-1) (FIG. 10C). These beneficial anti-fibrotic effects were translated into a significant reduction of hepatocyte dysfunction (reflected by the levels of serum-detectable transaminases) and an improved hepatic synthesis of albumin and proteins (FIG. 4D).

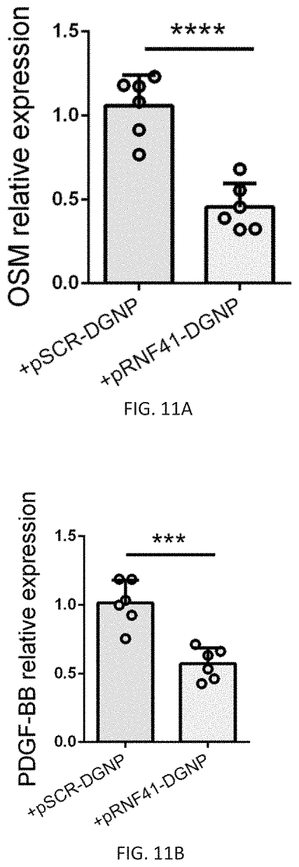

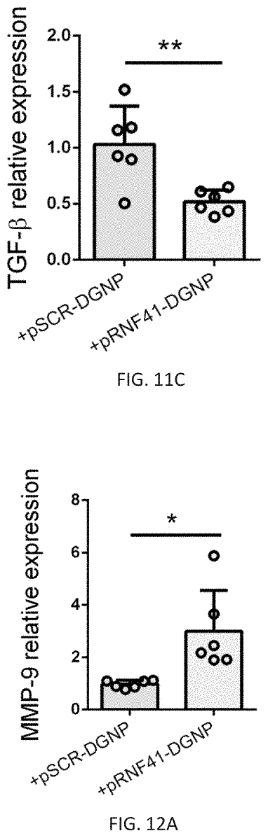

Inflammatory macrophages stimulate HSC activation and subsequent fiber production during liver fibrosis through the synthesis and release of agents such as oncostatin M (OSM), platelet derived growth factor-BB (PDGF-BB) and transforming growth factor-beta (TGF-β). Indicating that macrophage RNF41 hinders ECM excessive production in fibrosis in part though the downregulation of major macrophage-derived signals involved in HSC activation, it was found that pRNF41-DGNP promoted a significant reduction in hepatic OSM (FIG. 11A), PDGF-BB (FIG. 11B) and TGF-β expression (FIG. 11C). Indeed, RNF41 not only promoted the synthesis of HSC-inhibitory factors in macrophages but also macrophage overproduction of the collagenase MMP-9 to boost collagenous fiber digestion (FIG. 12A).

Fibrotic tracts limit spatially hepatocyte expansion. The drastic reduction in these collagenous chains in the liver of cirrhotic mice treated with pRNF41-DGNP was associated with an intense hepatic proliferative signal as highlighted by the increase in proliferating cell nuclear antigen (PCNA)-positive cells (FIG. 4E-1 and FIG. 4E-2). pRNF41 therapy essentially completely degraded fibrotic tracts; this means a drastic reduction in collagen (fibrosis) leaving a physiological parenchyma visually using Sirius Red Staining. This allowed that scarred cirrhotic liver from mice treated with pRNF41-DGNP displayed a remarkable liver mass repair (FIG. 12B). The absence of fibrotic septa allows hepatocyte expansion and tissue regeneration, therefore liver mass repair.

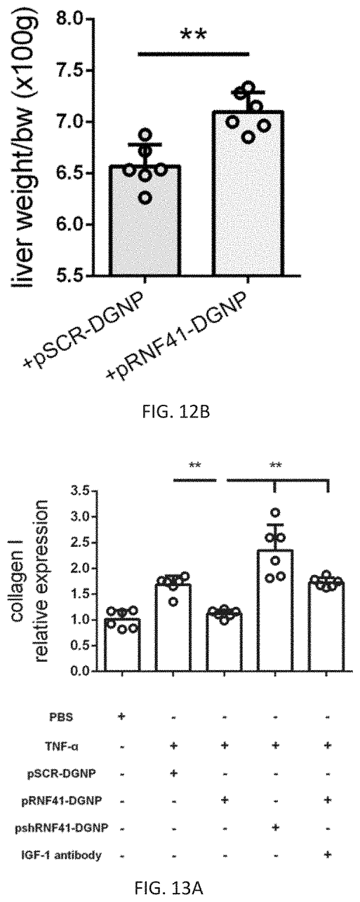

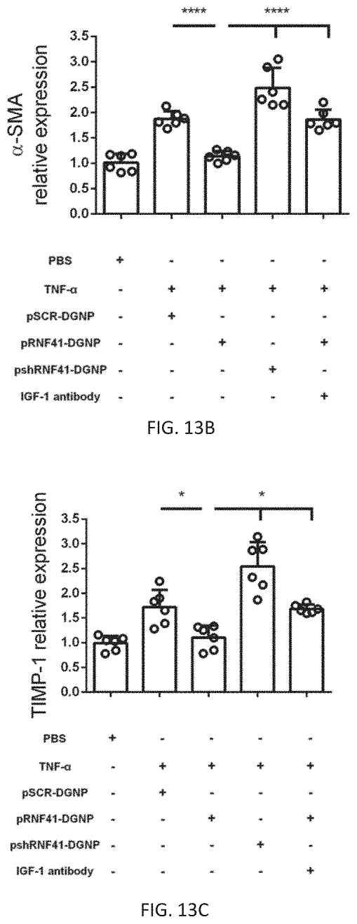

Interestingly, hepatocyte growth factor (HGF, the main hepatocyte proliferative factor) was not affected by pRNF41-DGNP in cirrhotic mice but insulin-growth factor 1 (IGF-1) expression was up-regulated in the liver of these animals (FIG. 4F). IGF-1 is related to hepatocyte proliferation and HSC inactivation. To determine if IGF-1 synthesized by macrophages treated with pRNF41-DGNP might be directly associated with the effects observed in hepatocyte proliferation and HSC activation, hepatocytes isolated from mouse livers with serum-starved conditioned media obtained from inflamed RAW 264.7 macrophages were stimulated with pRNF41-DGNP, pSCR-DGNP or DGNP containing a plasmid with an inhibitory shRNF41 (shRNF41-DGNP) in the presence or absence of a specific antibody against IGF-1. Only conditioned medium from macrophages treated with pRNF41-DGNP stimulated hepatocyte proliferation similar to FBS (10%), and this proliferative induction was drastically reduced with the addition of an antibody blocking IGF-1 effects (FIG. 4G). The proliferation induced by pRNF41 reaches 0.275 from the basal 0.20. IGF-1 antibody reduced that 0.275 to 0.215, almost basal levels. The effects of macrophage RNF41-induced synthesis of IGF-1 on LX-2 human HSC activation using the same conditioned media and experimental conditions was also tested. Conditioned medium from macrophages treated with TNF-α and pSCR-DGNP up-regulated the HSC expression of collagen I (FIG. 13A), α-SMA (FIG. 13B) and TIMP-1 (FIG. 13C), which was abolished by the treatment with pRNF41-DGNP and then recovered when IGF-1 was blocked with a specific antibody. pshRNF41 is blocking almost completely macrophage RNF41, and therefore exacerbating pro-fibrogenic and proinflammatory profile on these macrophages. Using conditioned media from pshRNF41 macrophages on hepatic stellate cells, we observe that hepatic stellate cells are overactivated producing more fibers and marker of activation TIMP1. Therefore, blockade of RNF41 stimulated factors released from macrophages to promote hepatic stellate cell activation and fibrosis. IGF-1 is mediating the antifibrotic properties stimulated by the presence of RNF41 and if blocked, hepatic stellate activation remains.

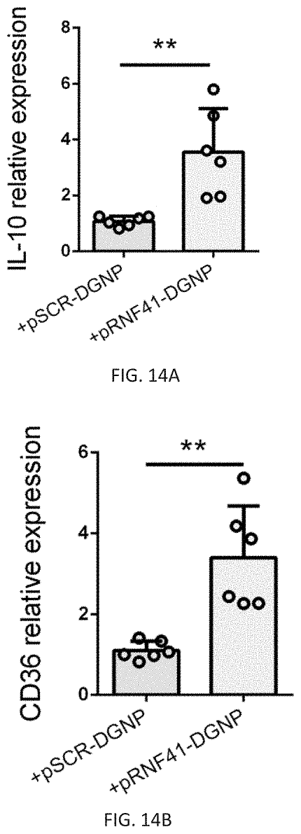

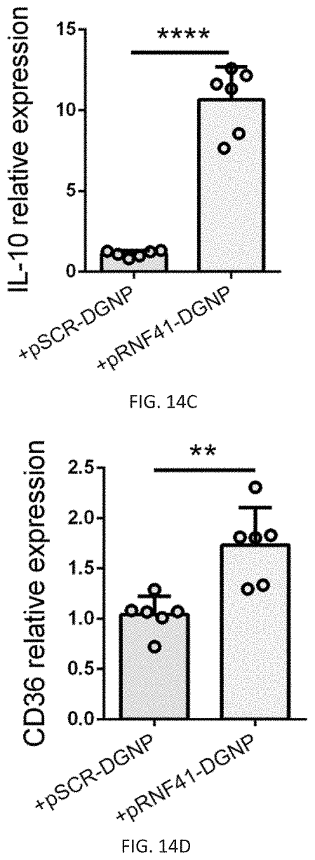

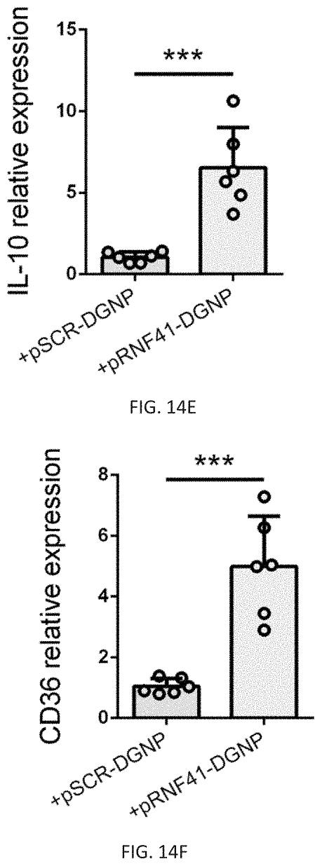

Macrophages displaying a M2-like phenotype are actively involved in hepatic fibrosis regression and tissue regeneration by means of influencing the response of HSC, endothelial cells and other immune cells to injury. To investigate whether RNF41 restoration was involved in the modulation of hepatic macrophage subsets, gene expression of M1-like and M2-like macrophage markers in cirrhotic livers from animals treated with either pSCR-DGNP or pRNF41-DGNP was quantified. Here, a quantitatively significant increase in M2 markers (ARG1, MRC1 and RETN1A) and a decrease in M1 markers (NOS2, COX-2 and IL-1β), denoting that this RNF41 restorative gene therapy switched pro-inflammatory M1-like to M2-like pro-regenerative macrophages in cirrhotic livers was shown (FIG. 4H). PPAR-γ activation triggers macrophages into alternative M2-like phenotype, which displays anti-inflammatory properties. To evaluate PPAR-γ activation in hepatic macrophages the expression of both downstream PPAR-γ target genes IL-10 and CD36 in cirrhotic livers from animals treated with either pSCR-DGNP or pRNF41-DGNP was quantified. pRNF41-DGNP stimulated the expression of IL-10 (FIG. 14A) and CD36 (FIG. 14B) in the cirrhotic liver, denoting an increased PPAR-γ activation.

### Example 5. Additional RNF41 Depletion in Hepatic Macrophages Aggravates Inflammation and Hepatic Damage and Reduces Survival

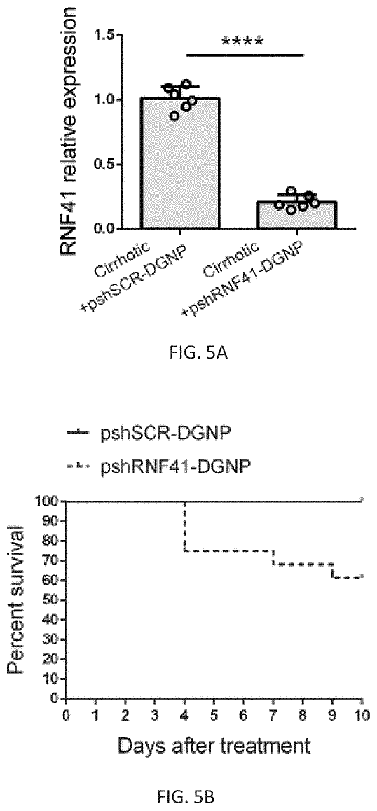

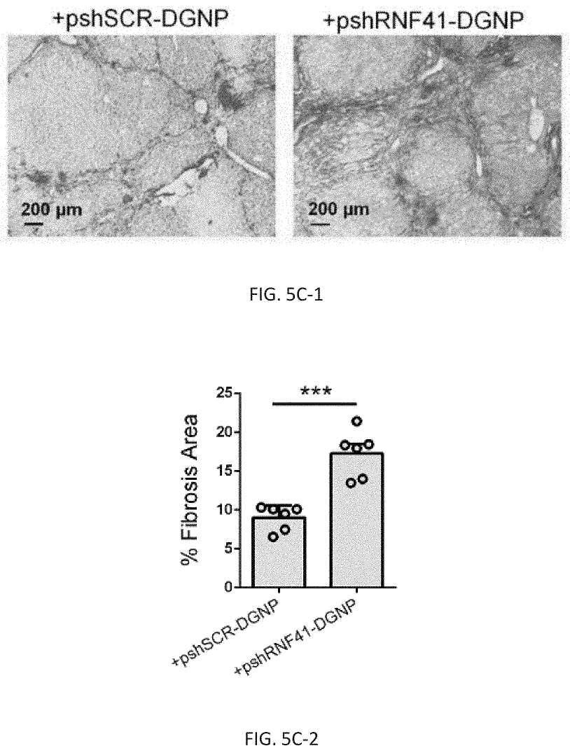

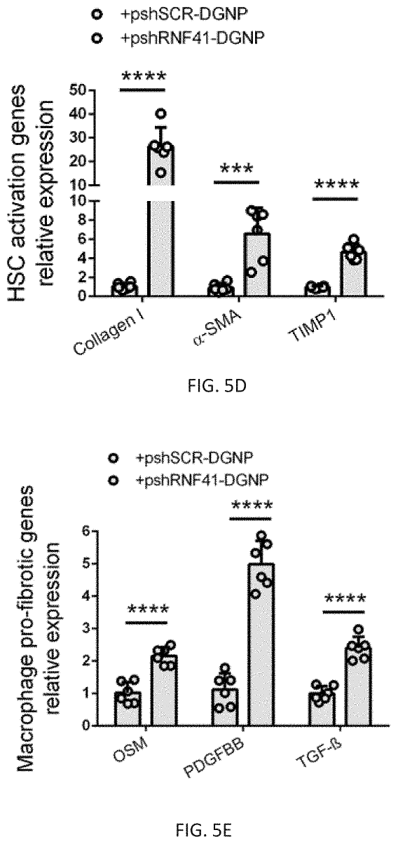

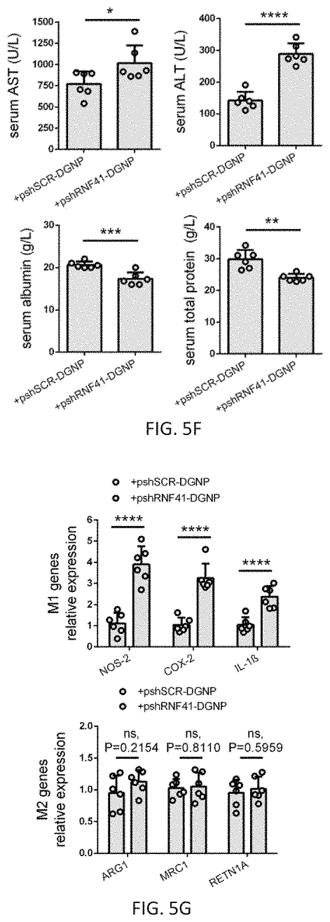

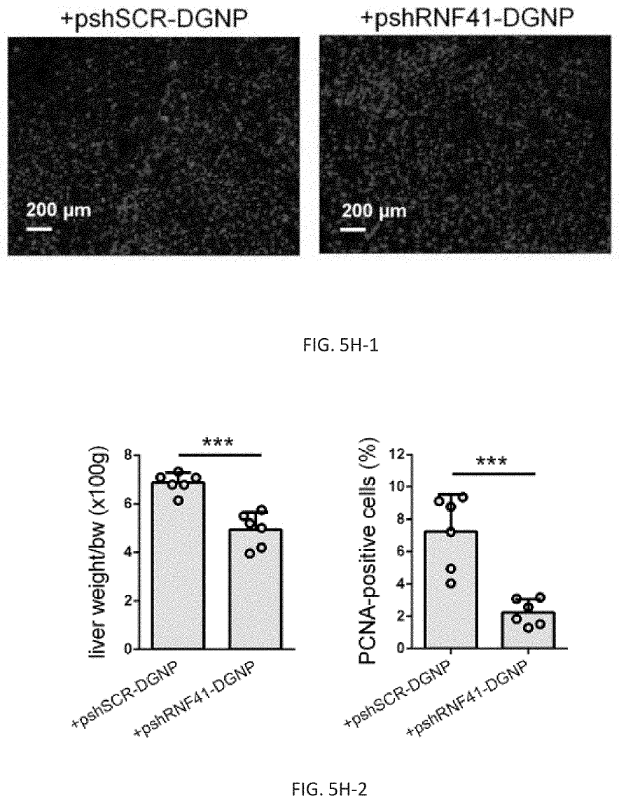

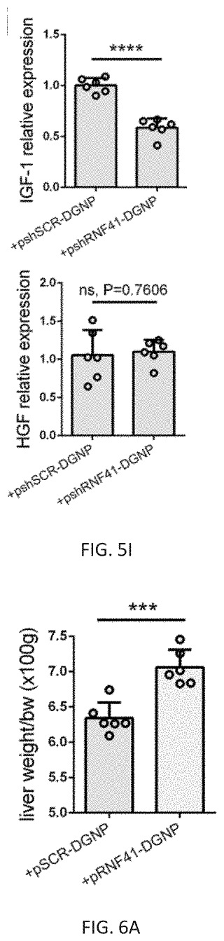

Either pshRNF41-DGNP or pshSCR-DGNP were intravenously administered to mice with liver cirrhosis every 3 days for 10 days. RNF41 expression was considerably reduced in isolated macrophages from cirrhotic mice treated with pshRNF41-DGNP (FIG. 5A). The first observable effect of RNF41 depletion was a significant decrease in survival (FIG. 5B). Collagen fiber staining revealed a devastating increment in fibrosis area in mice receiving pshRNF41-DGNP (FIG. 5C-1 and FIG. 5C-2). Hepatic area covered with collagen fibers in tissue slides was much higher in mice treated with pshRNF41. 20% of fibrosis area in mice is comparable to advanced cirrhosis in humans, and it was devastating to the point that many animals died. This was associated with an HSC hyperactivation, explained by the higher expression of collagen I, α-SMA and TIMP-1 (FIG. 5D). The amazing increase in HSC gene expression profiles is so dramatic because of the shRNF41 treatment in macrophages, HSC are those cells that secrete collagen and smooth muscle actin in fibrotic liver. Indeed, the treatment with pshRNF41-DGNP promoted a significant increase in hepatic expression of the macrophage-derived HSC activators OSM, PDGF-BB and TGF-β (FIG. 5E). pshRNF41 is blocking RNF41 so it was expected that if RNF41 recovery was antifibrotic, blockade of gene expression of RNF41 with pshRNF41 could be pro-fibrotic. Here, it was shown that macrophages can release factors that regulate inflammation and fibrosis and that RNF41 seems to control these factors upon inflammatory stimuli. If macrophage RNF41 decreases in any way, inflammation and fibrosis are found. Hepatocyte damage was also higher in cirrhotic animals treated with pshRNF41-DGNP, as reflected by the significant increment of serum-detectable transaminases and the further reduction of hepatic synthesis of albumin and proteins in comparison to cirrhotic mice receiving pshSCR-DGNP (FIG. 5F). The detrimental effects of pshRNF41-DGNP on hepatocyte function in cirrhotic mice were associated with a further increase in M1 macrophage-derived inflammatory cytokines and, therefore, with higher hepatic inflammation without affecting M2 macrophage genes (FIG. 5G). This exacerbated inflammation or cytokine storm resulted in a dampened hepatic proliferation and liver mass repair (FIG. 5H-1 and FIG. 5H-2). These effects in hepatocyte proliferation and HSC activation promoted by the depletion of macrophage RNF41 with pshSCR-DGNP were associated with a significant decrease in IGF-1 expression while no changes in HGF expression were observed (FIG. 5I).

### Example 6. Therapy with PRNF41-DGNP Induces Hepatic Regeneration after Hepatectomy

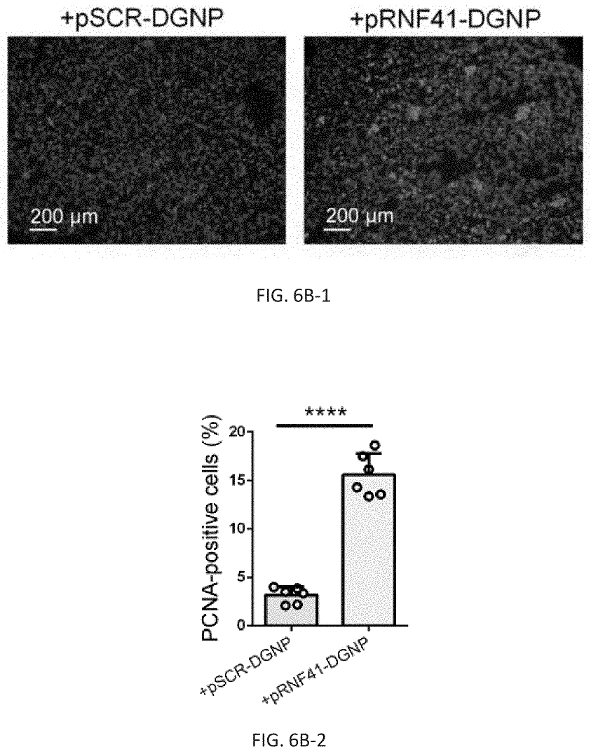

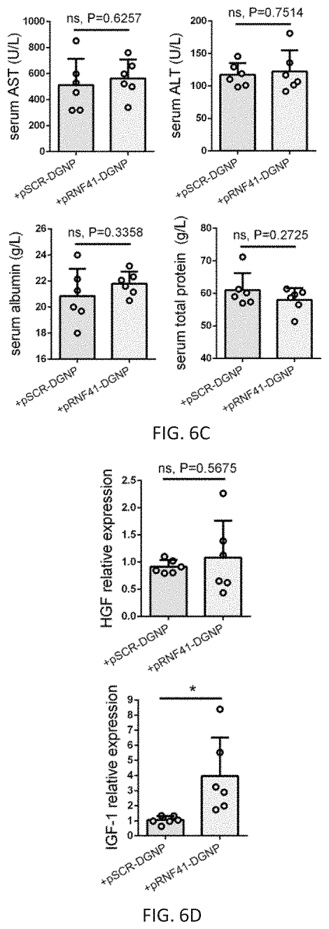

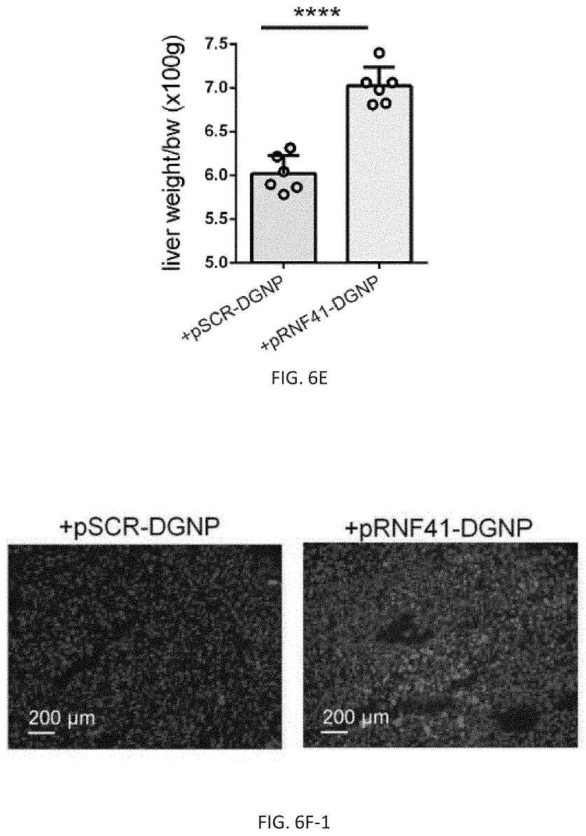

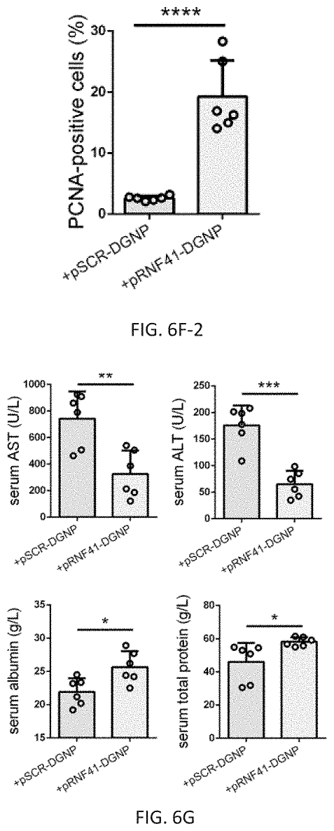

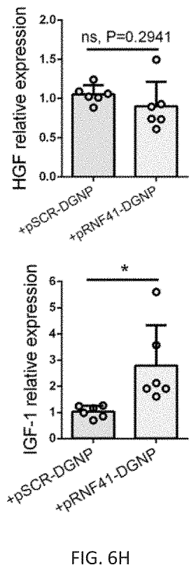

Example 6 was conducted to illustrate that increasing macrophage RNF41 could be also beneficial in the context of liver resection since hepatectomy and liver transplantation are the standard of care in patients with tumors of hepatic origin and end-stage liver disease, respectively. Administration of pRNF41-DGNP to healthy mice undergoing 70% hepatectomy showed a higher hepatic restoration than animals receiving pSCR-DGNP (FIG. 6A). This effect was associated with a much higher hepatic proliferative signal highlighted by the increase in PCNA-positive cells (FIG. 6B-1 and FIG. 6B-2). However, the treatment with pRNF41-DGNP did not reduce hepatocyte damage caused by liver resection, as no changes in serum transaminases, albumin or proteins were observed compared with animals receiving pSCR-DGNP (FIG. 6C). In line with the outcomes in cirrhotic mice shown in Example 4, hepatectomized mice receiving pRNF41-DGNP did not show up-regulation of HGF but did show higher expression of IGF-1 (FIG. 6D) concomitant with an increased expression of both hepatic PPAR-γ target genes IL-10 and CD36 (FIG. 14C and FIG. 14D).

Cirrhotic mice operated on 40% hepatectomy and administered pRNF41-DGNP also displayed a higher liver restoration rate than animals receiving pSCR-DGNP (FIG. 6E). This effect was also proportional with an elevated hepatic proliferative signal demonstrated by the significant rise in PCNA-positive cells (FIG. 6F-1 and FIG. 6F-2). Liver resection is performed to remove liver tumors, or to obtain liver mass for transplantation from a donor to recipient. The limit for safe resection may range from 20% to 30% (remnant liver to total liver volume). In patients with injured livers (e.g., cirrhosis, cholestasis, or steatosis), a preoperative assessment of the risk and the extent of the injury can be determined by suitable methods known in the art. Therefore, a percentage for liver resection may be around 70-80%. In this case, the treatment with pRNF41-DGNP did reduce hepatocyte damage assessed by serum transaminases, with a significant increase in serum albumin and proteins compared with animals receiving pSCR-DGNP (FIG. 6G). In agreement with the outcomes in cirrhotic mice shown in Example 4, hepatectomized mice treated with pRNF41-DGNP did not display up-regulation of HGF but did show higher expression of IGF-1 (FIG. 6H). The treatment with pRNF41-DGNP also promoted an augmented expression of both hepatic PPAR-γ target genes IL-10 and CD36 in hepatectomized cirrhotic animals (FIG. 14E and FIG. 14F).

### Example 7. Targeting Patients with Chronic Liver Disease and/or Other Diseases Characterized by Inflammation and Fibrosis

Further in vitro models of inflammation on isolated macrophages are contemplated to assess RNF41 gene and protein regulation further. Moreover, further experiments contemplate using macrophage-specific conditional knockout mice for RNF41 to further understanding of the pathophysiological implications of RNF41 in inflammation, fibrosis, and regeneration.

Together, this disclosure shows that RNF41 regulates the response of macrophages to restore homeostasis after tissue injury.

### REFERENCES

1. Asrani, S. K., Devarbhavi, H., Eaton, J. & Kamath, P. S. Burden of liver diseases in the world. J Hepatol 70, 151-171 (2019).
2. Melgar-Lesmes, P. & Edelman, E. R. Monocyte-endothelial cell interactions in the regulation of vascular sprouting and liver regeneration in mouse. J Hepatol 63, 917-925 (2015).
3. Melgar-Lesmes, P., Balcells, M. & Edelman, E. R. Implantation of healthy matrix-embedded endothelial cells rescues dysfunctional endothelium and ischaemic tissue in liver engraftment. Gut 66, 1297-1305 (2017).
4. Oro, D. et al. Cerium oxide nanoparticles reduce steatosis, portal hypertension and display anti-inflammatory properties in rats with liver fibrosis. J Hepatol 64, 691-698 (2016).
5. Cordoba-Jover, B. et al. Cerium oxide nanoparticles improve liver regeneration after acetaminophen-induced liver injury and partial hepatectomy in rats. J Nanobiotechnology 17, 112 (2019).
6. Forbes, S. J. & Rosenthal, N. Preparing the ground for tissue regeneration: from mechanism to therapy. Nat Med 20, 857-869 (2014).
7. Tsuchida, T. & Friedman, S. L. Mechanisms of hepatic stellate cell activation. Nat Rev Gastroenterol Hepatol 14, 397-411 (2017).
8. Trautwein, C., Friedman, S. L., Schuppan, D. & Pinzani, M. Hepatic fibrosis: Concept to treatment. J Hepatol 62, S15-24 (2015).
9. Sole, C. et al. Characterization of Inflammatory Response in Acute-on-Chronic Liver Failure and Relationship with Prognosis. Sci Rep 6, 32341 (2016).
10. Ramachandran, P. et al. Differential Ly-6C expression identifies the recruited macrophage phenotype, which orchestrates the regression of murine liver fibrosis. Proc Natl Acad Sci USA 109, E3186-3195 (2012).
11. Moroni, F. et al. Safety profile of autologous macrophage therapy for liver cirrhosis. Nat Med 25, 1560-1565 (2019).
12. Wauman, J., De Ceuninck, L., Vanderroost, N., Lievens, S. & Tavernier, J. RNF41 (Nrdp1) controls type 1 cytokine receptor degradation and ectodomain shedding. J Cell Sci 124, 921-932 (2011).
13. Wang, C. et al. The E3 ubiquitin ligase Nrdp1 ‘preferentially’ promotes TLR-mediated production of type I interferon. Nat Immunol 10, 744-752 (2009).
14. Ye, S. et al. The E3 ubiquitin ligase neuregulin receptor degradation protein 1 (Nrdp1) promotes M2 macrophage polarization by ubiquitinating and activating transcription factor CCAAT/enhancer-binding Protein beta (C/EBPbeta). J Biol Chem 287, 26740-26748 (2012).
15. Ruffell, D. et al. A CREB-C/EBPbeta cascade induces M2 macrophage-specific gene expression and promotes muscle injury repair. Proc Natl Acad Sci USA 106, 17475-17480 (2009).
16. Melgar-Lesmes, P. et al. Graphene-Dendrimer Nanostars for Targeted Macrophage Overexpression of Metalloproteinase 9 and Hepatic Fibrosis Precision Therapy. Nano Lett 18, 5839-5845 (2018).
17. Wu, X., Yen, L., Irwin, L., Sweeney, C. & Carraway, K. L., 3rd. Stabilization of the E3 ubiquitin ligase Nrdp1 by the deubiquitinating enzyme USP8. Mol Cell Biol 24, 7748-7757 (2004).
18. Liedtke, C. et al. Experimental liver fibrosis research: update on animal models, legal issues and translational aspects. Fibrogenesis Tissue Repair 6, 19 (2013).
19. Munoz-Luque, J. et al. Regression of fibrosis after chronic stimulation of cannabinoid CB2 receptor in cirrhotic rats. J Pharmacol Exp Ther 324, 475-483 (2008).
20. Connolly, M. K. et al. In liver fibrosis, dendritic cells govern hepatic inflammation in mice via TNF-alpha. J Clin Invest 119, 3213-3225 (2009).
21. Cao, Z., Wu, X., Yen, L., Sweeney, C. & Carraway, K. L., 3rd. Neuregulin-induced ErbB3 downregulation is mediated by a protein stability cascade involving the E3 ubiquitin ligase Nrdp1. Mol Cell Biol 27, 2180-2188 (2007).
22. Madge, L. A. & Pober, J. S. A phosphatidylinositol 3-kinase/Akt pathway, activated by tumor necrosis factor or interleukin-1, inhibits apoptosis but does not activate NFkappaB in human endothelial cells. J Biol Chem 275, 15458-15465 (2000).
23. Sabio, G. & Davis, R. J. TNF and MAP kinase signalling pathways. Semin Immunol 26, 237-245 (2014).
24. Conde, J., Oliva, N., Atilano, M., Song, H. S. & Artzi, N. Self-assembled RNA-triple-helix hydrogel scaffold for microRNA modulation in the tumour microenvironment. Nat Mater 15, 353-363 (2016).
25. Gustafson, H. H., Holt-Casper, D., Grainger, D. W. & Ghandehari, H. Nanoparticle Uptake: The Phagocyte Problem. Nano Today 10, 487-510 (2015).
26. Santos, J. L. et al. Receptor-mediated gene delivery using PAMAM dendrimers conjugated with peptides recognized by mesenchymal stem cells. Mol Pharm 7, 763-774 (2010).
27. Carroll, M. J., Kapur, A., Felder, M., Patankar, M. S. & Kreeger, P. K. M2 macrophages induce ovarian cancer cell proliferation via a heparin binding epidermal growth factor/matrix metalloproteinase 9 intercellular feedback loop. Oncotarget 7, 86608-86620 (2016).
28. Pradere, J. P. et al. Hepatic macrophages but not dendritic cells contribute to liver fibrosis by promoting the survival of activated hepatic stellate cells in mice. Hepatology 58, 1461-1473 (2013).
29. Reichenbach, V. et al. Adenoviral dominant-negative soluble PDGFRbeta improves hepatic collagen, systemic hemodynamics, and portal pressure in fibrotic rats. J Hepatol 57, 967-973 (2012).
30. Tonkin, J. et al. Monocyte/Macrophage-derived IGF-1 Orchestrates Murine Skeletal Muscle Regeneration and Modulates Autocrine Polarization. Mol Ther 23, 1189-1200 (2015).
31. Nishizawa, H. et al. IGF-I induces senescence of hepatic stellate cells and limits fibrosis in a p53-dependent manner. Sci Rep 6, 34605 (2016).
32. Das, A. et al. Monocyte and macrophage plasticity in tissue repair and regeneration. Am J Pathol 185, 2596-2606 (2015).
33. Bouhlel, M. A. et al. PPARgamma activation primes human monocytes into alternative M2 macrophages with anti-inflammatory properties. Cell Metab 6, 137-143 (2007).
34. Arkun, Y. Dynamic Modeling and Analysis of the Cross-Talk between Insulin/AKT and MAPK/ERK Signaling Pathways. PLoS One 11, e0149684 (2016).
35. Turowec, J. P. et al. Functional genomic characterization of a synthetic anti-HER3 antibody reveals a role for ubiquitination by RNF41 in the anti-proliferative response. J Biol Chem 294, 1396-1409 (2019).
36. De Ceuninck, L., Wauman, J., Masschaele, D., Peelman, F. & Tavernier, J. Reciprocal cross-regulation between RNF41 and USP8 controls cytokine receptor sorting and processing. J Cell Sci 126, 3770-3781 (2013).
37. Ramachandran, P. et al. Resolving the fibrotic niche of human liver cirrhosis at single-cell level. Nature 575, 512-518 (2019).
38. Krenkel, O. & Tacke, F. Liver macrophages in tissue homeostasis and disease. Nat Rev Immunol 17, 306-321 (2017).
39. Fabregat, I. et al. TGF-beta signalling and liver disease. FEBS J 283, 2219-2232 (2016).
40. Bonefeld, K. & Moller, S. Insulin-like growth factor-I and the liver. Liver Int 31, 911-919 (2011).
41. Lefterova, M. I. et al. PPARgamma and C/EBP factors orchestrate adipocyte biology via adjacent binding on a genome-wide scale. Genes Dev 22, 2941-2952 (2008).
42. Mitchell, C. & Willenbring, H. A reproducible and well-tolerated method for ⅔ partial hepatectomy in mice. Nat Protoc 3, 1167-1170 (2008).
43. Mederacke, I., Dapito, D. H., Affo, S., Uchinami, H. & Schwabe, R. F. High-yield and high-purity isolation of hepatic stellate cells from normal and fibrotic mouse livers. Nat Protoc 10, 305-315 (2015).
44. Ribera, J. et al. A small population of liver endothelial cells undergoes endothelial-to-mesenchymal transition in response to chronic liver injury. Am J Physiol Gastrointest Liver Physiol 313, G492-G504 (2017).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of inducing M2-like macrophage morphology in a macrophage, comprising contacting a macrophage with a composition, wherein the composition comprises a polynucleotide encoding a ring finger protein 41 (RNF41), under conditions whereby the composition enters the macrophage and RNF41 is expressed.

2. The method of claim 1, wherein the macrophage is a tumor-associated macrophage.

3. The method of claim 1, wherein the M2-like macrophage morphology consists of elevated expression of mannose receptor CD206, elevated production of matrix metalloproteinases, elevated collagenase activity, or a combination thereof.

4. The method of claim 1, wherein the macrophage has elevated anti-inflammatory factors, elevated anti-fibrotic factors, elevated pro-regenerative factors, or a combination thereof.

5. The method of claim 1, wherein the macrophage is in a subject, and the method comprises administering to the subject an effective amount of the composition, or a pharmaceutically acceptable salt thereof.

6. The method of claim 1, wherein the subject has, or is predisposed to have, tissue injury.

7. The method of claim 6, wherein the tissue is liver tissue, muscle tissue, lung tissue, spleen tissue, kidney tissue, a tissue of the mononuclear phagocyte system, or a combination thereof.

8. The method of claim 5, wherein the subject has chronic liver disease, liver failure, chronic liver inflammation, chronic hepatic fibrosis, cirrhosis, or a combination thereof.

9. The method of claim 5, wherein the subject has undergone a partial or complete hepatectomy, liver transplantation, or a combination thereof.

10. The method of claim 6, wherein tissue inflammation is reduced, tissue fibrosis is reduced, tissue repair is induced, or a combination thereof.

11. The method of claim 6, wherein hepatic fibrosis is reduced.

12. The method of claim 6, wherein hepatic regeneration is stimulated.

13. A method of promoting hepatic regeneration in a subject in need thereof, the method comprising administering to the subject an effective amount of a composition comprising a polynucleotide encoding ring finger protein 41 (RNF41).

14. The method of claim 13, wherein the polynucleotide encoding RNF41 is a plasmid, wherein plasmid further comprises a promoter, wherein the promoter is CD11b.

15. The method of claim 13, wherein the plasmid is operably linked to a graphite nanoparticle by at least one PAMAM generation 5 dendrimer.

16. The method of claim 13, wherein the subject has or is predisposed to having cardiovascular disease, diabetes, an auto-immune disease, allergic asthma, inflammatory bowel disease, chronic hepatic and/or renal disease, malignancy, Alzheimer's disease, or a combination thereof.

17. A composition comprising a polynucleotide encoding a ring finger protein 41 (RNF41), wherein the polynucleotide encoding RNF41 is a plasmid, and wherein the plasmid is operably linked to a graphite nanoparticle.

18. The composition of claim 17, wherein the plasmid further comprises a promoter, wherein the promoter is CD11b.

19. The composition of claim 17, wherein the plasmid is operably linked to the graphite nanoparticle.

20. The composition of claim 19, wherein the plasmid is operably linked to the graphite nanoparticle by at least one polyamidoamine (PAMAM) generation 5 dendrimer.
